(19) United States
(12) Patent Application Publication
(10) Pub. No.: US 2018/0208456 A1
(43) Pub. Date: Jul. 26, 2018

(54) PROGRAMMABLE, SELF ASSEMBLING PATCHED NANOPARTICLES, AND ASSOCIATED DEVICES, SYSTEMS AND METHODS

(71) Applicant: DNP123 Company, Altoona, IA (US)

(72) Inventors: Aaron T. Santos, Indianola, IA (US); Derek M. Lyons, Altoona, IA (US)

(73) Assignees: DNP123 Company, Altoona, IA (US); Simpson College, Indianola, IA (US)

(21) Appl. No.: 15/745,897

(22) PCT Filed: Jul. 21, 2016
(86) PCT No.: PCT/US16/43303

§ 371 Date: Jan. 18, 2018

Related U.S. Application Data
(60) Provisional application No. 62/195,175, filed on Jul. 21, 2015.

(51) Int. Cl. B81B 7/00; C07H 21/04; A61K 9/14; B81C 1/00; B22F 9/24; B22F 1/00

(52) U.S. Cl. CPC B81B 7/0003; C07H 21/04; A61K 9/14; B81C 1/00007; H01L 27/11807; B22F 1/0044; B22F 1/0062; B81C 2201/0149; B22F 9/24

(57) ABSTRACT

The present invention generally relates to nanofabrication and, in some embodiments, to methods of synthesizing selectively binding patched nanoparticles and the devices that can be made from them. In some embodiments, the invention relates to methods of assembling arbitrarily shaped structures from patched nanocubes and the devices and uses that follow. For example, nanocube building blocks may be patched by stamping their faces with a selectively binding chemical species (e.g. DNA, antibody-antigen pairs, etc.), or by using self-assembly to attach to the nanocubes multiple selectively binding patch species whose immiscibility can be preprogrammed. Arbitrarily shaped structures can then be designed and assembled by deciding which faces will be bonded to each other in some target structure and combining nanocubes that have selectively binding patches on those faces. Other aspects of the invention are also directed to methods of making such nanocubes or other nanoparticles, methods of forming such nanocubes.

### RELATED APPLICATIONS

This application claims the benefit of U.S. Provisional Patent Application Ser. No. 62/195,175, filed Jul. 21, 2015, incorporated herein by reference in its entirety.

### FIELD

The present invention generally relates to nanofabrication and, in some embodiments, to methods of synthesizing selectively binding patched nanoparticles and devices that can be made from them.

### BACKGROUND

Any discussion of the prior art throughout the specifications should in no way be considered as an admission that such prior art is widely known or forms part of common general knowledge in the field.

Researchers have grown adept at synthesizing spheres, cubes, prisms, tubes, lattices of particles, and a variety of other highly symmetric, monodisperse systems of identical particles whose dimensions are less than one micrometer. However, synthesizing more complicated, asymmetrical shapes of these dimensions is very difficult and overly complex using currently available methods. While we lathes, 3D printers, and a variety of other shape molding methods can be used to create complex shapes in the macroscale, current techniques cannot create a complete set of all possible geometric shapes with nanometer dimensions.

For example, the use of DNA oligomers to provide programmability has been realized by many groups and has inspired the entire field of DNA origami. DNA origami is a technology that utilizes DNA programmability to achieve asymmetric, complex nanostructures. This technology suffers from several major drawbacks. First, only fairly simple structures can assemble before DNA mismatches occur and inhibit the formation of superstructures. As such, complicated structures form in relatively low yield. Second, the DNA origami does not possess the functionality inherent to nanoparticles.

In addition, DNA coated nanoparticles have been synthesized for almost 20 years. While these structures can be made in a variety of shapes, (e.g. spheres, cylinders, cubes) they typically have only one or at most two different species of DNA coating the surface of a single nanoparticle. Further, it is difficult to control the relative locations of different DNA patches on the nanoparticle surface using currently available techniques. Consequently, it is difficult to program the assembly of complex structures in any easily generalizable way.

The creation of nanocubes with two species (i.e. hydrophilic and hydrophobic) of patches has been demonstrated by the stamping of patches onto nanocubes. This assembly method is incapable of creating arbitrarily shaped structures, because only a limited variety of shapes can be made with only two species of patches. Moreover, it is limited to structures made from a low number of nanocubes as the hydrophobic interactions between nanocubes are relatively weak. While certain embodiments of nanocubes patched with multiple selectively patches have been shown to be theoretically stable under ideal conditions, these theoretical results suffer several shortcomings. First of all, they are theoretical. They do not provide any experimental methods of actually assembling the patched nanoparticles; they only show that the particles would be thermodynamically stable if a method could be found to synthesize them. Such theoretical studies also assume tunable immiscibility between the patches without explaining how such a situation would actually arise experimentally. They also do not provide any explanation of how such patches could be selective, what materials may be used to create such patches, or how such patches could be selectively stamped only on specific locations of the nanocubes.

### SUMMARY

The present invention generally relates to nanofabrication and, in some embodiments, to methods of synthesizing selectively binding patched nanoparticles and the devices that can be made from them. The subject matter of the present invention involves, in some cases, interrelated products, alternative solutions to a particular problem, and/or a plurality of different uses of one or more systems and/or articles.

For example, some embodiments of the present invention are generally directed to devices, systems, and methods involving the creation of programmable building blocks that may be used to build arbitrarily shaped nanostructures, for instance, via self-assembly. In some embodiments, these methods can be used to create patched nanoparticles, on which there may exist three or more selectively binding patches. Various systems and methods describe cases where unique patches of DNA exist on nanoparticle faces is discussed in detail, but these should be regarded as exemplary only, and other embodiments of the invention are applicable to patches of other selectively binding materials in any location on the nanoparticle, including the vertices and edges.

Certain embodiments discussed herein are directed to various methods of assembling nanoparticles with three or more selectively binding patches and various methods of assembling arbitrarily shaped structures from these patched nanoparticles. In certain aspects, disclosed are patched nanoparticle assembly methods comprising stamping the faces with three or more species of selectively binding chemical patches. In another embodiment, disclosed are patched nanoparticle assembly methods comprising combining nanocubes in solution with three or more species of selectively binding chemicals that contain a sequence of regions with predetermined miscible properties. In another embodiment, disclosed are methods of synthesizing arbitrarily shaped nanostructures comprising connecting some combination of nanoparticles synthesized using the methods above in solution and allowing the complimentary selectively binding patches on different particles to bind. These methods may allow the formation of structures that, in various embodiments, (1) can be preprogrammed to have any arbitrary shaped desired, (2) feature simple design rules, (3) exhibit nanoparticle functionality (e.g. electrical, optical, catalytic properties, etc.), and/or (4) can be extended to larger structures.

According to another aspect, the present invention is generally directed to a composition. In some embodiments, the composition includes a plurality of nanoparticles. In certain embodiments, the composition is a superstructure, e.g., comprising nanoparticles.

In one set of embodiments, the composition comprises a superstructure comprising at least three nanoparticles, joined in face-to-face contact to form the superstructure. In some embodiments, each face-to-face contact of the superstructure is defined by a binding interaction between the respective contacting nanoparticles. In some instances, each of the binding interaction within the superstructure of nanoparticles comprises no more than 10% of the total binding interactions within the superstructure of nanoparticles.

The composition, in another set of embodiments, comprises a superstructure comprising at least three nanoparticles bonded together via specific binding interactions. In some cases, each of the binding interactions within the superstructure of nanoparticles comprises no more than 10% of the total binding interactions within the superstructure of nanoparticles.

In still another set of embodiments, the composition includes a stable superstructure comprising at least three nanoparticles, where at least two of the nanoparticles are not in contact with each other within the superstructure.

According to yet another set of embodiments, the composition comprises a stable superstructure formed from a plurality of nanoparticles, where no more than 50% of the nanoparticles forming the superstructure are identical.

In another set of embodiments, the composition comprises a plurality of superstructures formed from nanoparticles bound together by noncovalent interactions. In some cases, at least 50% of the superstructures comprise at least three nanoparticles and are indistinguishable.

Still another set of embodiments is generally directed to a plurality of superstructures, where the superstructures are formed from nanoparticles joined in face-to-face contact to form the superstructures. In certain cases, at least 50% of the superstructures comprise at least three nanoparticles and are indistinguishable.

In yet another set of embodiments, the composition is generally directed to a suspension comprising a plurality of stable superstructures formed from nanoparticles. In some cases, at least 30% of the superstructures within the suspension comprise at least three nanoparticles and are indistinguishable.

In one set of embodiments, the composition comprises a first nanoparticle, comprising a first face comprising a first binding partner, a second face comprising a second binding partner, and a third face comprising a third binding partner, and a second nanoparticle, comprising a first face comprising a binding partner. In certain instances, the binding partner of the second nanoparticle is able to specifically bind to the first binding partner of the first nanoparticle without specifically binding to the second or third binding partners.

According to another set of embodiments, the composition comprises a plurality of nanoparticles, comprising at least first and second nanoparticles each comprising faces. In some embodiments, the faces of each of the first and second nanoparticles have different arrangements of binding partners. In certain cases, only one face of the first nanoparticle and one face of the second nanoparticle have binding partners that can specifically bind to each other.

Yet another set of embodiments is generally directed to an electronic circuit comprising a conductive pathway defined by a plurality of polyhedral nanoparticles joined in face-to-face contact to form the conductive pathway.

Another set of embodiments is generally directed to a superstructure having an interior space. The superstructure may be formed from a plurality of polyhedral nanoparticles.

Still another set of embodiments is generally directed to a plurality of nanoparticles positioned to form a superstructure. According to certain embodiments, the superstructure may have at least one surface defined by the faces of at least some of the nanoparticles forming the superstructure.

Yet another set of embodiments is generally directed to a sheet formed from a plurality of nanocubes. In some cases, the sheet has a thickness defined by the thickness of a single nanocube, two nanocubes, three nanocubes, or more nanocubes.

Another aspect of the invention is generally directed to a method. In certain cases, the method includes methods of forming nanoparticles; adding patches, binding entities, or the like to nanoparticles; and/or assembling nanoparticles to form superstructures, e.g., as discussed herein. Some embodiments of the invention are also generally directed to articles made from these methods, or kits or methods of using such articles.

In some embodiments, the method comprises applying a first coating to a first face of a plurality of nanoparticles comprising faces without applying the coating to a second face of the nanoparticles, and applying a second coating to the second face of the nanoparticles without applying the coating to the first face of the nanoparticles. In some cases, the method includes enriching the plurality of nanoparticles in nanoparticles having a specific arrangement of the first and second faces.

Another set of embodiments is generally directed to a method of synthesizing a patched nanocube comprising stamping the faces with three or more species of selectively binding patches. Still another set of embodiments is generally directed to a method of synthesizing a patched nanocube comprising combining the nanocubes in solution with three or more species of selectively binding chemicals that contain a sequence of regions with different miscible properties.

In another set of embodiments, the method comprises synthesizing a patched nanocube comprising stamping the faces of a nanocube with three or more species of selectively binding patches. Yet another set of embodiments is generally directed to a method of synthesizing a superstructure comprising patched nanocubes, comprising combining nanocubes in solution with three or more species of selectively binding chemicals that contain a sequence of regions with different miscible properties.

The method, in another set of embodiments, is generally directed to a method of synthesizing a superstructure. In some cases, the method comprises combining nanostructures in solution with three or more species of selectively binding chemicals that contain a sequence of regions with different miscible properties. In still another set of embodiments, the method is generally directed to a method of synthesizing a patched nanostructure comprising stamping the faces of a nanostructure with three or more species of selectively binding patches.

In another aspect, the present invention encompasses methods of making one or more of the embodiments described herein, for example, nanoparticles exhibiting selective binding. In still another aspect, the present invention encompasses methods of using one or more of the embodiments described herein, for example, nanoparticles exhibiting selective binding.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1F are schematics of some possible binding arrangements of three nanocubes;

FIGS. 2A-2B are schematics of the assembly design for a certain embodiment of an arbitrarily shaped structure;

FIG. 3 is a schematic of nanocube assembly, according to certain embodiments;

FIGS. 4A-4C are schematics of the formation of immiscible patches, according to certain embodiments;

FIGS. 5A-5B show exemplary chemical structures of polymers, according to certain embodiments;

FIG. 6 shows additional exemplary chemical structures of polymers, according to certain embodiments;

FIG. 7 is a schematic representation of self-assembly of patches on a nanoparticle surface, according to certain embodiments;

FIG. 8 is a schematic representation of possible configurations a given chemical species may assume when confined on a nanocube's faces, in accordance with certain embodiments;

FIGS. 9A-9D are schematic representations of a patch stamping procedure, according to certain embodiments;

FIGS. 10A-10C are schematic representations of nanoparticle assembly, according to certain embodiments;

FIG. 11 illustrates nanocube binding, according to certain embodiments;

FIGS. 12A-12B are schematics of nanocube binding, according to various embodiments;

FIG. 13A-13C are schematic representations of a direction-specific selectively binding patch, according to certain embodiments;

FIG. 14A-14B are schematic representations of another direction-specific selectively binding patch, according to certain embodiments;

FIGS. 15A-15C are schematic representations of controllable flexibility nanowire assembly, according to certain embodiments;

FIGS. 16A-16C are schematic representations of controllable conductivity nanowire assembly, according to certain embodiments;

FIGS. 17A-17B are a schematic representations of nanosheet assembly, according to certain embodiments;

FIGS. 18A-18C are other schematic representations of controllable flexibility nanosheet assembly, according to certain embodiments;

FIGS. 19A-19C are schematic representations of porous nanosheet assembly, according to certain embodiments;

FIGS. 20A-20C are schematic representations of nanohelix assembly, according to certain embodiments;

FIGS. 21A-21B are a schematic representations of transistor assembly, according to certain embodiments;

FIGS. 22A-22D are schematic representations of the assembly and operation of a drug delivery device, according to certain embodiments;

FIGS. 23A-23B are schematic representations of the assembly and operation of a molecular recognition device, according to certain embodiments; and

FIG. 24 shows the shift in the absorbance spectrum between unhybridized and hybridized nanocubes, in yet another embodiment.

### DETAILED DESCRIPTION

The present invention generally relates to nanofabrication and, in some embodiments, to methods of synthesizing selectively binding patched nanoparticles and the devices that can be made from them. In some embodiments, the invention relates to methods of assembling arbitrarily shaped structures from patched nanocubes and the devices and uses that follow. For example, nanocube building blocks may be patched by stamping their faces with a selectively binding chemical species (e.g. DNA, antibody-antigen pairs, etc.), or by using self-assembly to attach to the nanocubes multiple selectively binding patch species whose immiscibility can be preprogrammed. Arbitrarily shaped structures can then be designed and assembled by deciding which faces will be bonded to each other in some target structure and combining nanocubes that have selectively binding patches on those faces. Other aspects of the invention are also directed to methods of making such nanocubes or other nanoparticles, methods of forming such nanocubes or other nanoparticles into devices, devices formed from such nanocubes or other nanoparticles, kits including such nanocubes, nanoparticles, or devices, or the like.

Certain aspects of the presently disclosed devices, systems, and methods use “building blocks” to build complex arbitrarily shaped superstructures via self-assembly or other techniques described herein. In some cases, such assembly methods may be thought of as being programmable or predetermined, e.g., as such the final superstructure may be determined based on the initial design of binding of the various building blocks with each other.

These building blocks may utilize nanocubes (or other nanoparticles), on which three or more selectively binding chemical “patch” species cover each face, partially or completely. Typically, a “patch” will be present predominately on one face (or in some cases, more than one face), but will not be present in significant amounts on other faces Some embodiments of the presently disclosed techniques also utilize such “patching” to assemble the nanostructures into superstructures, which can be used in a wide variety of applications, including those discussed herein.

The “building blocks” or nanoparticles that are assembled by some of the methods may have various advantages. For instance, some embodiments are directed to the self-assembly of arbitrarily-shaped superstructures. These may be formed using the simple cubical shape of nanocubes and/or multiple selectively binding patches on various faces of the nanocubes or other nanoparticles, which may be, for example, face-centered, programmable, stackable, etc.

Incorporating both the cubical or other stackable geometry and a plurality of selectively binding patches, can allow a variety of substantial and transformative improvements. For example, by incorporating more than two patches, programmability can be added, e.g., to allow the assembly of any arbitrary or designed superstructure from a plurality of nanocubes or other nanoparticles. Patterned programmable selectively binding chemicals in patches on the nanoparticles may be achieved in some embodiments, which may be useful for the assembly of superstructures, e.g., into various devices.

For instance, in some embodiments, programmability may allow one to pre-design the shape of the final target superstructure. The geometry of the nanocubes or other nanoparticles may, in some cases, allow for face-to-face binding. The flat faces can be conjoined nearly parallel to each other, making designing target superstructures simple, because the nanoparticles can be bound flush against each other, and can be aligned on a straight-line rectangular grid, or in other predictable formats, depending on the nanoparticles. This geometry may permit the design and assembly of larger superstructures.

Thus, such programmability may allow a superstructure to be defined on the basis of the ability of various nanoparticles s to bind, e.g., in specific configurations or arrangements, thereby forming the superstructure. Such design may occur in some cases even before the nanoparticles are synthesized. In some cases, such programmability may allow only one, or a relatively small number, of final superstructures to be designed and assembled from the nanoparticles. For instance, after assembly, at least 50% or more of the superstructures may share essentially identical configurations of nanoparticles that from the superstructures.

As a non-limiting example, the formation of a superstructure from a plurality of nanocubes is now discussed. It should be understood that nanocubes are discussed herein for ease of presentation and understanding only, but that the invention is not limited to only nanocubes. In other embodiments as discussed herein, other nanoparticles may also be used, in addition to and/or instead of nanocubes.

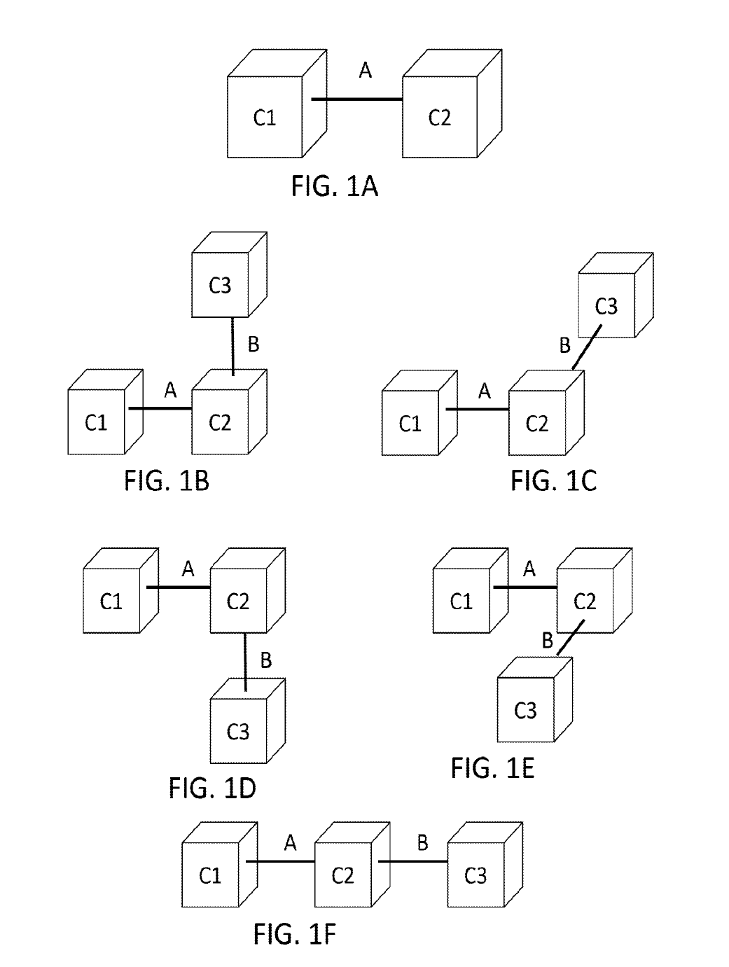

In one set of embodiments, nanocubes (or other nanoparticles) may be produced whose faces contain a “patch,” e.g., of a programmable selectively binding chemical such as is discussed herein. Each of the faces of the nanocubes may be independently controlled to have a patch (or lack thereof), and different faces of the nanocube may independently have the same or different patches. This may allow for the ability to design superstructures of a vast number of different shapes. Thus, for instance, for a nanocube, each of the 6 sides can be patched, e.g., with a selectively binding chemical. In FIG. 1A, as a specific example, a set of cubes (C1) can be synthesized such that each face is covered by a single DNA sequence, with no two faces having the same sequence. A second set of cubes (C2) can be synthesized in the same manner, except that one face contains a DNA sequence complementary to another sequence on the first set of nanocubes. The connection between these faces can be called A, as illustrated in FIG. 1A.

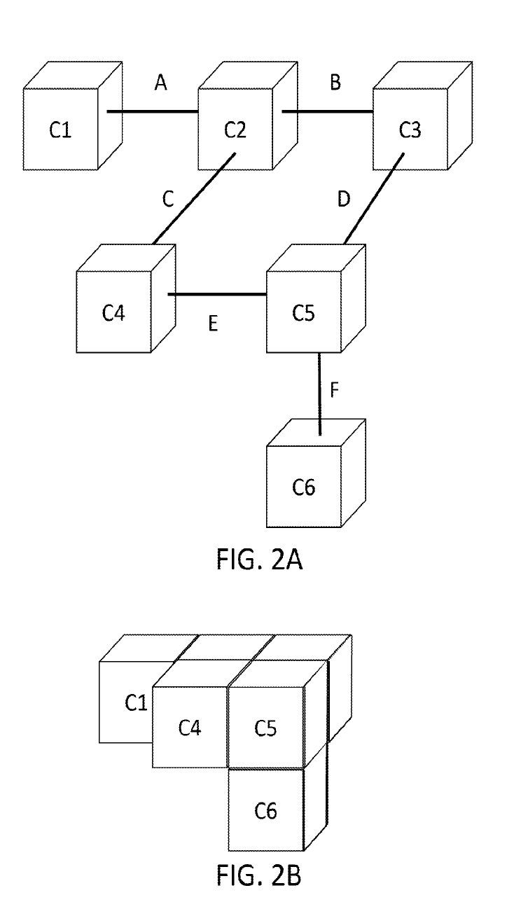

A third set of cubes (C3) may also be synthesized, such that one of its faces contains a complimentary sequence to a face on C2, such that cubes C2 and C3 form a connection labeled by B. The C2 face on which connection B is made can be at any of five non-A locations (the four faces adjacent to the A side as well as the side opposite the A side), thereby allowing the programmable formation of five distinct geometries as illustrated in FIGS. 1B through 1F. Note that there is no suitable binding connection between C1 and C3. Iterating this binding method of connecting faces containing programmable selectively binding chemical patches allows for the creation of programmed superstructures of any number of nanocubes (e.g., C1, C2, C3, C4, C5, C6) in any desired arbitrary shape depending on the arrangement of the face connectors (e.g., A, B, C, D, E and F, etc.) as is illustrated in FIG. 2 (shown with connections A, B, C, D, E, and F in FIG. 2A, and with the connections hidden in FIG. 2B).

Because single-stranded DNA will typically only bind with its complementary strand, these various nanocubes bind together at complementary faces only. This allows for control which faces will bind together to form specific dimers of nanocubes. To form superstructures, this process can be repeated or iterated by synthesizing many nanocubes, each with its own unique DNA patches (e.g., shown for example in FIG. 1). In these embodiments, each nanocube can be thought of as a “pixel” or a “voxel” within a larger superstructure, as is shown in FIG. 2, for example, and such nanocubes may be assembled together into a two-dimensional or three-dimensional shape.

It should be understood that the configuration of nanocubes shown in FIGS. 1 and 2 is by way of explanation only, and in other embodiments, other superstructures may be formed, e.g., using nanocubes or other nanoparticles such as those discussed herein.

Accordingly, certain aspects of the invention are directed to nanoparticles. Such nanoparticles may be readily obtained commercially, and/or synthesized as discussed herein. In one embodiment, the nanoparticles may be nanocubes. A nanocube typically is substantially cube-shaped, although in reality, such nanocubes are not expected to be mathematically-perfect cubes. In practice, the dimensions and/or angles of such nanocubes may accordingly vary somewhat from the ideal mathematical cube. For instance, the nanocubes may have a height, length, or width that varies less than 20 nm, less than 15 nm, less than 10 nm, or less than 5 nm of the other dimensions, and/or the angles defining the nanocube may not be precisely 90°, but may be between 80° and 100°, or between 85° and 95°, etc.

In addition to nanocubes, the nanoparticles may have other shapes as well, such as cylinders, plates, prisms, rectangular solids (which may or may not have a square face, and which may be orthogonal or may be skewed or non-orthogonal in 2 or 3 dimensions), or other platonic solids (e.g., tetrahedron, octahedron, dodecahedron, or icosahedron). Thus, in further embodiments, a variety of other faceted nanoparticle shapes can be synthesized, including tetrahedrons, octahedrons, and icosahedrons, to name a few. In some cases, the nanoparticles have a shape such that they may be stacked together without gaps, e.g., such as cubes, rhombic dodecahedrons, truncated octahedrons, tetrahedron/octahedron honeycombs, or other 3-dimensional tessellation shapes. The nanoparticles may also have semiregular or irregular shapes in some embodiments. In certain embodiments, the outer surface of nanoparticle is defined by substantially flat planar surfaces, e.g., as in a polyhedron. There may be any suitable number of flat surfaces defining the nanoparticle, e.g., 4, 5, 6, 7, 8, 9, 10, 11, 12, 13, 14, 15, 16, etc. The faces may independently be of the same or different shapes and/or sizes, and may be regular or irregular. In some cases, the nanoparticles have at least one pair of opposed sides that are parallel to each other, and in certain cases, the nanoparticles may have two, three, or more pairs of opposed sides that are parallel to each other.

A nanocube or other nanoparticle typically has a largest internal dimension of less than about 1 micrometer, e.g., such that it is measured on the order of nanometers. For example, in some cases, the nanoparticle may have a largest internal dimension of less than about 900 nm, less than about 800 nm, less than about 700 nm, less than about 600 nm, less than about 500 nm, less than about 400 nm, less than about 300 nm, less than about 200 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

The nanoparticles may be formed from any suitable material. Examples of nanoparticle compositions useful in various embodiments of the invention include metals (e.g. gold, silver, platinum, copper, and iron, etc.), semiconductors (e.g. silicon, silicon, copper selenide, copper oxide, cesium oxide, etc.), magnetic materials (e.g., iron oxide), or the like. Combinations of these are also possible, e.g., gold-silver nanoparticles, gold-copper nanoparticles, etc. In some cases, the nanoparticle comprises an alloy of 2, 3, or more metals. One non-limiting example of a gold-copper nanoparticle is described in Example 9. Methods of making nanoparticles with different compositions and/or geometries are known in the art.

For example, in one set of embodiments, nanoparticles may be created using polyol-mediated synthesis. Polyol mediated synthesis of nanoparticles may be initiated in some cases by reduction of a metal salt into a metal ion at high temperature. A capping agent may interact with a nanoparticle surface to influence the nanoparticle size and shape. In various embodiments, ethylene glycol, a polyol, can act as both the reducing agent and the capping agent, in addition to capping agents (e.g. polyvinylpyrrolidone and cetyltrimethylammonium bromide (CTAB)) and reducing agents (e.g. sodium hydrosulfide and ascorbic acid).

In some embodiments, the composition of the nanoparticle can be determined by the identity of the metal salt used. For example, silver nitrate can be used for synthesis of silver nanoparticles and gold chloride can be used for synthesis of gold nanoparticles. Other metal nanoparticles such as those discussed herein can be prepared using corresponding metal salts, e.g., metal chlorides or metal nitrates.

The size and shape of the nanoparticle can be controlled in various embodiments by controlling reaction conditions like the reaction time, identity of the reaction components (e.g. capping and reducing agents), and/or the concentration of components in the reaction. For example, the size of the nanoparticles can be controlled by quenching a synthesis reaction at a desired time. In some embodiments, the shape of the nanoparticles may be controlled by controlling the concentrations of capping agents and/or reducing agents. For example, gold nanocubes can be formed using low CTAB and high ascorbic acid concentrations, whereas high CTAB and low ascorbic acid concentrations may favor formation of octahedral shapes in certain embodiments.

In one set of exemplary embodiments, gold nanoparticles are utilized. For example, gold, in the form of a salt, may be dissolved in solvent and reduced by a reducing agent. The size and morphology of the gold nanoparticles may be controlled by the addition of capping agents to the reaction. The capping agent can be attached to the surface of the gold nanoparticle, kinetically or thermodynamically inhibiting additional atoms from joining the crystal. Gold nanoparticles can be purified by a variety of methodologies, including centrifugation, column chromatography, and gel electrophoresis.

In some cases, more than one nanoparticle may be present, including any combination of any of those discussed herein. For instance, if more than one type of nanoparticle is present, the nanoparticles may independently differ on the basis of shape, size, material, or the like, and/or combinations thereof. For example, there may be two, three, or more sizes of nanocubes present, and/or there may be a variety of different shapes of nanoparticles present (e.g., nanotetrahedrons and/or nanoctahedrons), and/or there may be a variety of nanoparticles comprising different materials that are present.

The nanoparticles that are present may have a narrow size distribution in some embodiments. For instance, the nanoparticles may have a distribution such that less than about 30%, less than about 20%, less than about 10%, less than about 5% of the nanoparticles have a largest internal dimension that is greater than 120% or less than 80%, or greater than 110% or less than 90%, of the average largest internal dimension of all of the nanoparticles.

As discussed, the nanoparticles may include one or more “patches” on one or more faces in various aspects. For instance, a face of a nanoparticle may be modified with a chemical able to selectively bind other chemicals, e.g., attached to the faces of other nanoparticles. The face may thus be described as having a selectively binding chemical or a “patch.” The patches may then be used to assemble nanoparticles together into superstructures.

Patches may be present on one or more faces of a nanoparticle, e.g., to 2, 3, 4, 5, 6, 7, 8, or more faces of a nanoparticle. The patches on each face of the nanoparticle may independently be the same or different. In addition, as discussed above, different nanoparticles may have different patches on them, e.g., to allow for the creation of more complex structures using nanoparticles.

Thus, at least some of the patches may be used to bind or attach the nanoparticles to other nanoparticles, e.g., to form a superstructure of nanoparticles. The patches may be used to establish face-to-face binding or contact, e.g., between different nanoparticles, and the alignment of nanoparticles may be centered or off-centered in some cases. In some cases, the patches may be relatively unique, e.g., a patch may be able to specifically bind to only one (or a small number) of other patches within the superstructure. Such specificity may allow only a small number of binding interactions between nanoparticles to occur, thereby allowing a specific superstructure to form. For example, out of all of the binding interactions forming a superstructure, each of the binding interactions may form no more than 50%, no more than 40%, no more than 30%, no more than 20%, no more than 10%, no more than 5%, or no more than 2% of all of the binding interactions that form the superstructure. Different binding interactions may be non-interchangeable with each other, e.g., such that only certain combinations of binding partners (and thus, only certain nanoparticles are able to stably contact each other). In some cases, each binding interaction within a superstructure of nanoparticles is unique.

A patch may independently cover all, or only a portion of, a face of a nanoparticle such as a nanocube. For instance, the patch may cover at least 10%, at least 20%, at least 30%, at least 40%, at least 50%, at least 60%, at least 70%, at least 80%, at least 90%, or substantially the entire face and/or no more than 90%, no more than 80%, no more than 70%, no more than 60%, no more than 50%, no more than 40%, no more than 30%, no more than 20%, or no more than 10% of the available surface area on the face of a nanoparticle such as a nanocube. Different faces of the nanocube may independently exhibit different amounts of coverage (or no coverage) by a patch, and different faces of a nanoparticle may exhibit the same or different patches, for instance, by being identical or different chemically, recognizing different binding partners, etc.

For brevity, some embodiments will be referred to herein as a “patching system,” though this is not meant to restrict the embodiments to one specific modality, as the associated devices and methods are also contemplated. Accordingly, in some embodiments of the invention, the disclosed patching methods segregate multiple selectively binding chemical patches on separate faces of nanocubes. “Patchy particles” (meaning particles on which at least one well-defined patch generates an anisotropic, directional interaction with other particles) can be used in certain embodiments.

In some cases, patches may be created by binding partners, which may be specific or non-specific. In some embodiments, a patch is able to only bind to one other specific patch within the superstructure without being able to stably bind to other, incompatible patches within the superstructure.

Because of its simple, sequence-dependent self-assembly characteristics, DNA is useful as a binding partner for a patch, e.g., as discussed herein. However, it should be understood that DNA is described here as one example, and other binding systems (or combinations of binding systems) may be used in other embodiments, such as discussed below. In some embodiments, for example, DNA can be segregated on the faces of a nanocube or other nanoparticle, which may simplify programmability or assembly, etc., as discussed herein.

The term “binding partner” or “binding chemical” generally refers to a molecule that can undergo binding with a particular partner, typically to a significantly higher degree than to other molecules, e.g., specific binding. For instance, the binding interaction between specific binding partners may be at least 10×, 100×, or 1000× greater than for any other binding partners that are present. In some cases, the binding between the binding partners may be essentially irreversible. Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen, etc. The binding interactions between binding partners may be, for example, hydrogen bonds, van der Waals forces, hydrophobic interactions, covalent coupling, or the like.

Thus, as other examples besides DNA hybridization (and/or hybridization of other nucleic acids), suitable patch systems include lock and key protein interactions such as avidin-biotin or enzyme-substrate interactions, antibody-antigen pairs, covalent coupling interactions, hydrophilic/hydrophobic/fluorinated interactions, and the like. Examples of some of these are discussed herein. As noted above, DNA may be particularly useful because of its simple programmable sequence-dependent binding rules, but the invention is not limited to only DNA patches. In addition, in some embodiments, more than one such system may be used, e.g., within the same patch, within different patches on the same nanoparticle, on different nanoparticles, or the like.

In one set of embodiments, different nucleic acid strands may be attached to various faces of a nanoparticle, which may be used to form unique patches on some or all of the faces of the nanoparticle. The nucleic acid strands may include, DNA, RNA, PNA, XNA, and/or any suitable combination of these and or other suitable polymers, and may comprise naturally-occurring bases and/or non-naturally-occurring bases. In some cases, due to the specificity of unique nucleic acid strands with each other, selective binding may be achieved between different patches on different nanoparticles. The nucleic acid strands may have any suitable number of nucleotides, and different patches may have nucleic acid strands with the same or different numbers of nucleotides. As non-limiting examples, the nucleic acid strands may include at least 6, at least 7, at least 10, at least 12, at least 15, at least 20, at least 25, at least 30, at least 35, at least 40, at least 45, at least 50, at least 55, at least 60, at least 70, at least 80, at least 90, or at least 100 nucleotides, which may be suitable produce a large number of relatively unique patches. As an illustrative example, using only the 4 naturally-occurring nucleotides, a DNA nucleic acid strand with 10 nucleotides would have 4¹⁰=1,048,576 combinations available (although not all of them need be used).

In one set of embodiments, the miscibility of the patches may be different. Such miscibilities may be controlled, for example, by using moieties having different patterns of hydrophilicities/hydrophobicities. For instance, unique patches may be created on the faces of a nanoparticle using unique miscibilities on each face having a patch. Based on such miscibilities, binding partners having compatible miscibilities would be able to bind to the face while binding partners having incompatible miscibilities would be unable to bind to the face. In this way, unique patches may be created on some or all of the faces of the nanoparticle.

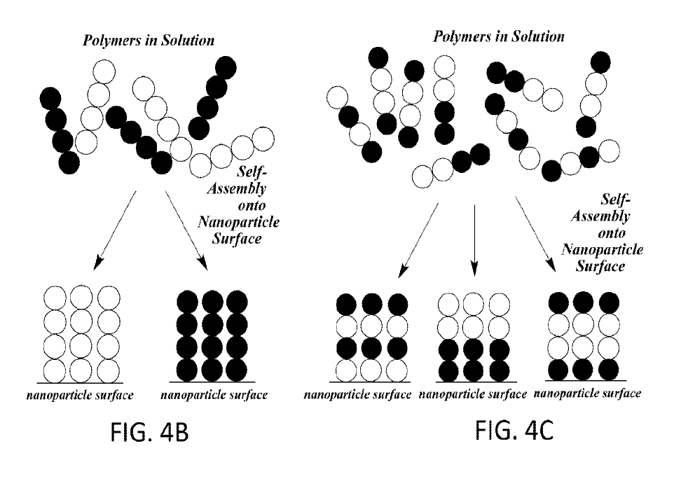

In some cases, miscibilities for the faces of a nanoparticle may be created using polymers having a variety of hydrophilic and/or hydrophobic groups, e.g., in a defined sequence. It should be understood that “hydrophilic” and “hydrophobic” groups are generally used in a relative sense with respect to miscibilities, i.e., hydrophilic groups generally prefer to associate with other hydrophilic groups rather than hydrophobic groups and vice versa, in such manner, a series of different hydrophilic groups and hydrophobic groups positioned within a polymer (e.g., as represented by white and black spheres in FIG. 4A) may define a miscibility for a polymer. It should also be understood that other interactions between hydrophilic/hydrophobic interactions may be used in other embodiments to define various miscibilities of a polymer; for example, such miscibilities may be defined by charged moieties within the polymer.

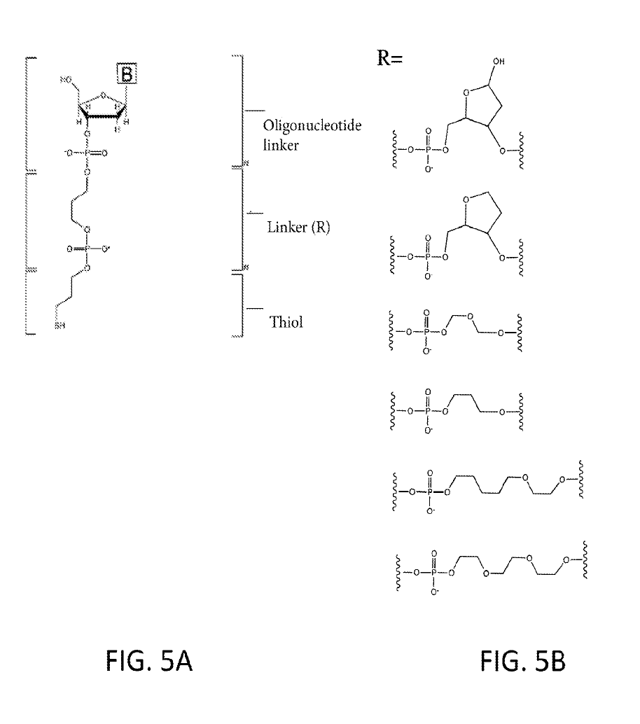

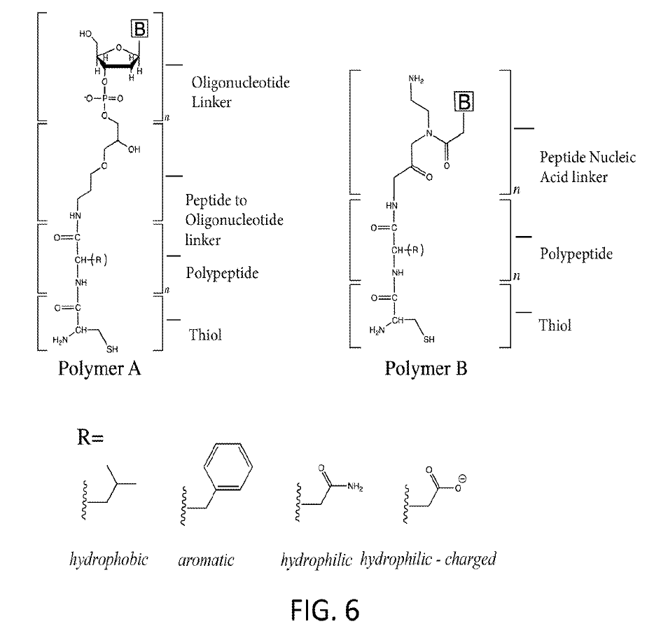

FIGS. 5-6 depict examples of embodiments of chemical structures of polymers comprising chemical moieties, for example, to control miscibilities. The polymers may be synthesized, for example, by chemically coupling monomers together to create patterns of chemical functionalities. The polymers in these examples may include a moiety (e.g. a thiol group) that bonds to the nanoparticle surface on one terminal end and a linker on the other end that displays chemically selective patch. For the sake of example, “B” in these figures may represent any of the five canonical nitrogenous bases found in nucleic acid polymers (i.e., adenine, thymine, cytosine, uracil, or guanine). “n” denotes the number of single monomer units that are repeated to build a polymer. “R” represents any type of chemical functionality used to provide chemical interactions between polymers. These examples represent the types of chemical functionalities useful for chemical interactions between polymers, but are not an inclusive list.

FIG. 5A depicts a polymer synthesized using phosphoramidite methodology to chemically couple the monomers. The linker region incorporates patterns of monomers with varying degrees of immiscible chemical properties (e.g. hydrophobicity, hydrogen/covalent/ionic bonding, etc.). FIG. 5B shows general non-limiting examples of varying chemical functionalities incorporated into the polymer at positions represented by “R.”

Non-limiting examples of hydrophilic and hydrophobic groups are shown in FIG. 6. The groups may be present within the backbone structure of the polymer and/or as side or pendant groups, in various embodiments. FIG. 6 provides a non-limiting example of a polymer synthesized using amide coupling chemical methodologies standard in peptide synthesis. Amino acid monomers can provide the patterning of chemical functionality useful for chemical interactions between polymers. The amino acid cysteine may provide the thiol moiety for linking the polymer to the nanoparticle. Polymer A in FIG. 6 shows an example of a peptide based polymer with a nucleic acid sequence attached by a peptide to oligonucleotide linker moiety. Polymer B in FIG. 6 incorporates the nitrogenous bases within a peptide nucleic acid based monomer, eliminating the need for a peptide to oligonucleotide linker moiety. Non-limiting examples of varying chemical functionalities incorporated into the polymer at positions represented by “R” are all of the canonical amino acid chemical functionalities (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), in addition to non-canonical functionalities ranging in hydrophobicity from hydrophobic hydrocarbons and halogenated compounds to hydrophilic, anionic and cationic chemical functionalities.

Representative examples of hydrophobic functionalities are hydrocarbons in the form of straight, branched, or cyclic structures with potential for varying degrees of unsaturation. Hexyl, 2-methyl-pentyl, trans-2-hexenyl, and cyclohexyl are representative hydrocarbon “R” groups. Aromatic functionalities can represent the “R” group, like phenyl or napthyl groups. Halogenated functionalities like tri-fluoromethyl can be incorporated in the “R” group. Hydrophilic functionalities can be non-ionic or ionic. Representative functionalities including ethers, esters, alcohols, acetals, amines, amides, aldehydes, ketones, nitriles, carboxylic acids, sulfates, sulfonates, phosphates, phosphonates, and nitro groups can be incorporated into the “R” group as ethylene glycol or butanenitrile, for example. Absence of an “R” group may be represented by a hydrogen or unsaturation. These examples represent the types of chemical functionalities useful for chemical interactions between polymers, but are not an inclusive list.

The polymer may include any suitable number of hydrophilic and hydrophobic groups, e.g., to form unique miscibilities suitable for attaching suitable binding partners to a face of a nanoparticle. In some cases, there may be at least 3, at least 4, at least 5, at least 6, at least 7, at least 10, at least 12, at least 15, at least 20, at least 25, at least 30, at least 35, at least 40, at least 45, at least 50, at least 55, at least 60, at least 70, at least 80, at least 90, or at least 100 such groups present. Such numbers may allow for relatively large numbers of unique miscibilities to be generated. For example, in a system comprising a polymer that can include a hydrophilic portion or a hydrophobic portion, 3 monomers would allow 2³=8 possibilities, while 10 monomers would allow 210=10²⁴ possibilities. In such fashion, relatively large numbers of unique patches may be used within a plurality of nanoparticles to build up a superstructure.

The following is a non-limiting example of how such patch systems may be used to assemble nanoparticles into a superstructure. In this example, six distinct chemical binding patches may be added to each of the six faces of a plurality of nanocubes. For purposes of illustration only, it is helpful to imagine these chemical patches as specific sequences of ssDNA, though the technique is generally applicable to other chemical species including, for example, antibody-antigen pairs or other patch techniques discussed herein. By way of explanation, the ssDNA used in this example for a nanocube will be referred to as A, B, C, D, E, and F. Since all sequences of unmodified ssDNA have similar hydrophobicity, each face will be uniformly coated in a mixture of all six sequences. To induce separation between the six different ssDNA sequences on the surface, the miscibility of the ssDNA may be modified. One approach involves diversifying the chemical functionality, e.g. hydrophobicity, of the linker on the ssDNA ligands. For example, in one embodiment, the head of the DNA ligands may be modified with a short sequence of alternating hydrophilic-hydrophobic moieties. When these ligands bind to surface, the alignment of alternating hydrophilic-hydrophobic regions within the ligand head induces a repulsive interaction between dissimilar species, similar to the repulsion between oil and water. By adjusting the size and sequence of the hydrophilic-hydrophobic regions, multiple patches with a programmable sequence of hydrophobicity may be created. Because of the different patterns of hydrophobicity contained within these two molecules, distinct patches of either all ssDNA-A or all ssDNA-B may be created. Similarly, by controlling the pattern of hydrophilic and hydrophobic regions, the entire surface of the cube may be coated with different distinct patches of ssDNA on each side.

FIGS. 4A-4C depict the self-assembly of patches on a nanoparticle surface using a polymer with patterns of miscible and immiscible monomers in this example. In FIG. 4A, chemical interactions of monomers are shown. The white and black spheres represent different types of monomers. Monomers of the same type interact favorably and attract, while dissimilar monomers interact unfavorably and repel. In FIG. 4B, the mixing of multiple polymers constructed from only one type of similar chemical functionalities in solution or suspension may cause demixing on the surfaces of nanoparticles that results in a nanoparticle surface coated in one of only two possible polymers. In FIG. 4C, polymers with multiple types of chemical functionalities patterned in different sequences are constructed, which allows for patch formation by the demixing of immiscible species.

FIG. 4 shows self-assembly of patches on a nanoparticle surface using a polymer with patterns of miscible and immiscible monomers. FIG. 4A shows chemical interactions of monomers. White and black spheres represent different types of monomers. Monomers of the same type interact favorably and attract, while dissimilar monomers interact unfavorably and repel. FIG. 4B shows that mixing multiple polymers constructed from only one type or similar chemical functionalities in solution or suspension causes demixing on the surfaces of nanoparticles that results in a nanoparticle surface coated in one of only two possible polymers. FIG. 4C shows that constructing polymers with multiple types of chemical functionalities in patterns of different chemical functionalities allows for programmable assembly controlled by the miscible/immiscible sequence.

Various methods of forming patches on the surfaces of nanocubes or other nanoparticles are disclosed herein, in various aspects. As an illustrative non-limiting example, one method of selectively binding a molecule to a face of a nanoparticle is provided here, using cap exchange, such as is described in Example 9.

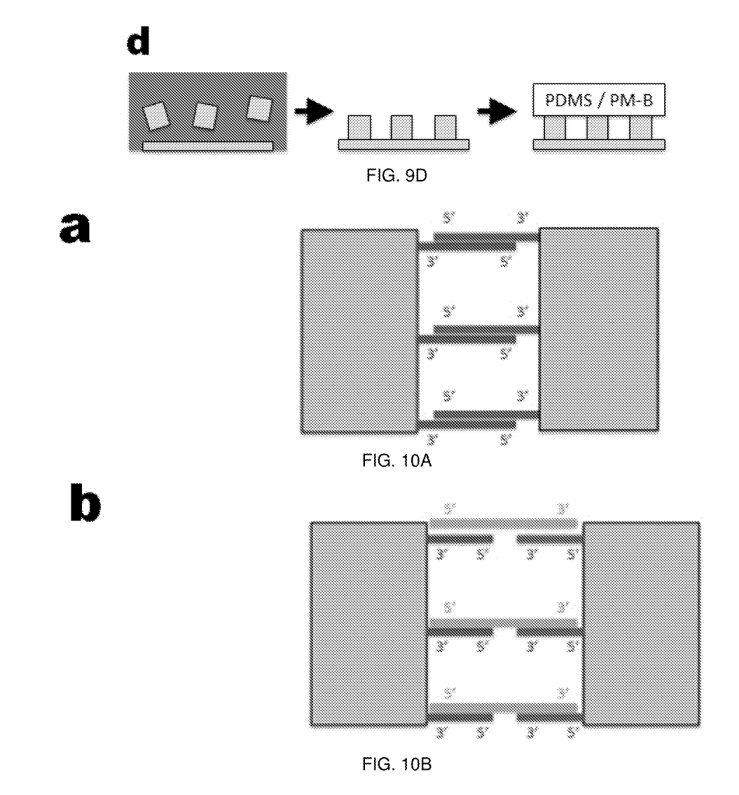

In another embodiment, for example, a suspension of nanoparticles such as nanocubes (e.g., produced or acquired as discussed herein) may be deposited onto a substrate, which may be atomically smooth in some cases, e.g. mica, a silicon wafer, etc., as illustrated in FIG. 9A. The substrate may be printed with a PDMS stamp inked with a patch molecule A (PM-A) as shown in FIG. 9B. For purposes of illustration, the patches may comprise DNA, antibody/antigen, or other binding partners as discussed herein. After coating the top face with the patch, the stamp can be removed and the nanoparticles can be immersed, e.g., in a suspension or solution containing a second patch molecule B (PM-B), which covers the uncoated sides of the nanoparticles as illustrated in FIG. 9C. After the sides have been coated, PM-B can be removed, and the system may be removed from the surface. This may be accomplished by, for example, sonicating the substrate in a bath, or by other suitable techniques. The nanoparticles may then be immersed in a solution or suspension containing a third patch molecule (PM-C). Sonication or other suitable techniques may be used to remove the nanoparticles from the substrate, exposing the bottoms of the nanoparticles, which will then be coated in PM-C. This may be used, for example, to create nanoparticles with patches A and C on opposite faces of the nanoparticles, and B patches on the remaining faces.

In yet another embodiment, from FIG. 9B, the PM-A coated nanoparticles may be sonicated in solution or suspension. The solvent may then be removed and the nanoparticles again dried on the surface, which may influence the probability of coating certain faces of the nanoparticle. For example, for a nanocube, by choosing a flat surface that interacts unfavorably with patch A, patch A may be on top at most ⅙^{th} of the time, on bottom ⅙^{th} of the time, and on its side at least ⅔^{rds} of the time. Choosing a flat surface with an affinity for certain patch molecules may result in significantly higher yields. For instance, by printing a PDMS stamp inked with PM-B, patches may be positioned on adjacent faces of the nanostructure. In FIG. 9D, steps such as those described above may be reiterated in some embodiments to produce unique patches on one or more (or all) faces of the nanoparticles. The nanoparticles can also be purified as discussed herein.

In another embodiment, nanoparticles may be formed using stamping. For example, in some cases, patches are made by first depositing nanoparticles such as nanocubes on a surface and stamping a selectively binding patch molecule on the upward face. In some cases, the nanoparticles may be immersed in a solution or suspension containing a second selectively binding patch molecule that may coat the unstamped sides. In another embodiment, the nanoparticles may be re-suspended, deposited again on a flat surface, and stamped again with a different selectively binding chemical species. The procedure can be iterated until at least three of the sides of the nanoparticles are patched. Multiple embodiments and improvements to this method include, but are not limited to, re-suspending the nanoparticles in a solution or suspension containing another species of patch molecule to patch multiple faces simultaneously, blocking faces with a weakly binding patch molecule that can be replaced later in the synthesis, using the flat surface to block the addition of chemical patches, using various chemically modified flat surfaces to improve the yield, etc.

In another set of embodiments, a cap exchange with multiple selectively binding species with different immiscibilities may be used. By way of example, consider inserting chemically modified DNA nucleotides into DNA ligands. Phase separation may occur on a nanoparticle's surface occurs when the ligands are immiscible. The head of the DNA ligands may be modified, for example, with a short sequence of alternating hydrophilic-hydrophobic moieties. When the ligands bind to a tightly packed particle surface, the forced alignment of alternating hydrophilic-hydrophobic regions within the ligand head group can induce dissimilar ligands to phase separate on the surface. By adjusting the size and sequence of the hydrophilic-hydrophobic regions, one can create multiple patches. In order to generate at least six distinct patches (one for each cubic face), three hydrophilic-hydrophobic “-mer” units may be added to the headgroup of the DNA. By assembling multiple patches of different DNA sequences on the nanoparticle, the superstructure assembly instructions may be encoded directly onto the nanoparticle surface.

In order to assemble the nanoparticles into arbitrarily shaped superstructures, it may be useful some embodiments to controllably position selectively binding patches on the faces of the nanoparticles. To accomplish this, in one set of embodiments, entropic effects may be used. These effects, due to the anisotropic curvature of faceted nanoparticles, induce preferential alignment of sterically bulky ligands on the edges and vertices of faceted particles. The patching system increases the degree of “bulkiness” by adding branched groups to the ligand. These bulkier ligands preferentially align along the edges and vertices. Accordingly, the patching system allows for the creation and sorting of nanoparticles having specific binding regions on each of the faces, thereby allowing for the specific arrangement of nanoparticle superstructures, as is described herein.

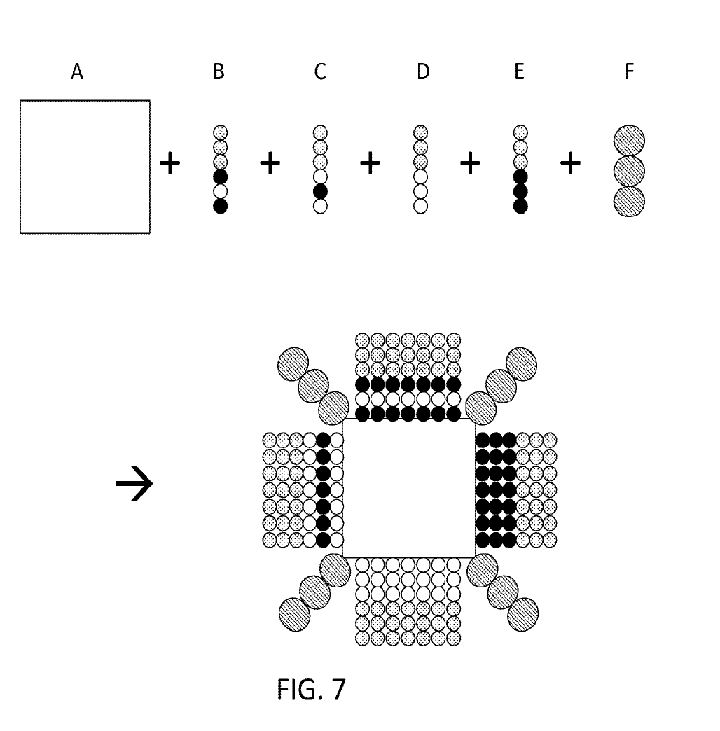

In some embodiments, segregation of patches on the faces of nanoparticles may be achieved using curvature-induced differences in conformational ligand entropy to position selectively binding chemical patches on the faces of the nanocubes or other nanoparticles. Since substrate curvature dictates the assembly of SAMs, the anisotropic curvature of faceted particles may provide a way to control surface assembly. Entropic effects, due to the anisotropic curvature of faceted nanoparticles, can induce preferential alignment of sterically bulky ligands on the edges and vertices of faceted particles. However, the degree of “bulkiness” may be increased in some embodiments by adding branched groups to the ligand. When two ligands attach to the surface of a faceted nanoparticle, the bulkier of the ligand species can preferentially align along the edges and vertices. For example, FIG. 7 depicts ligand separation on a faceted nanocube. In this exemplary depiction, two immiscible ligands form patches on the faces of a nanocube when in the presence of a third bulky ligand. This is in contrast to the disordered state one expects to occur on both spherical particles and faceted particles with ligands of equal thickness. The effect is pronounced in polyhedra like tetrahedrons and cubes where the facets connect at sharp angles.

For instance, in the example of FIG. 7, patch formation may occur when immiscible chemical species (represented here by B, C, D, and E) are added to solution or suspension containing nanocubes A. In the presence of a third bulkier patch molecule F, the immiscible patches may be controlled by F such that the patches are centered on the faces of the cube. Thus, when multiple thin immiscible ligands plus one additional sterically bulky ligand attach to the surface of a faceted particle, the bulky ligand may in some embodiments “corrals” the immiscible ligands, isolating them on separate faces. This may be used to form spontaneously self-assembled asymmetric selectively binding chemical patches on the surfaces of nanocube building blocks. When allowed to self-assemble, these building blocks can be used to form complex arbitrarily shaped superstructures in various embodiments.

In some cases, the synthesis of patched nanoparticles may produce nanoparticles in which the arrangement of the patches is random. For instance, some cubes may have a face with patch A adjacent to a face containing patch B, while other cubes will have patch A and patch B on opposite sides of the cube. In order to use the nanoparticles as effective building blocks for larger structures, in some cases, the different nanoparticles may be isolated or separated. This can be achieved through a variety of methods well known in the art, including but not limited to electrophoresis, column chromatography, and centrifugation. Accordingly, in one set of embodiments, the nanoparticles may be separated or enriched to produce nanoparticles having the desired arrangement of patches.

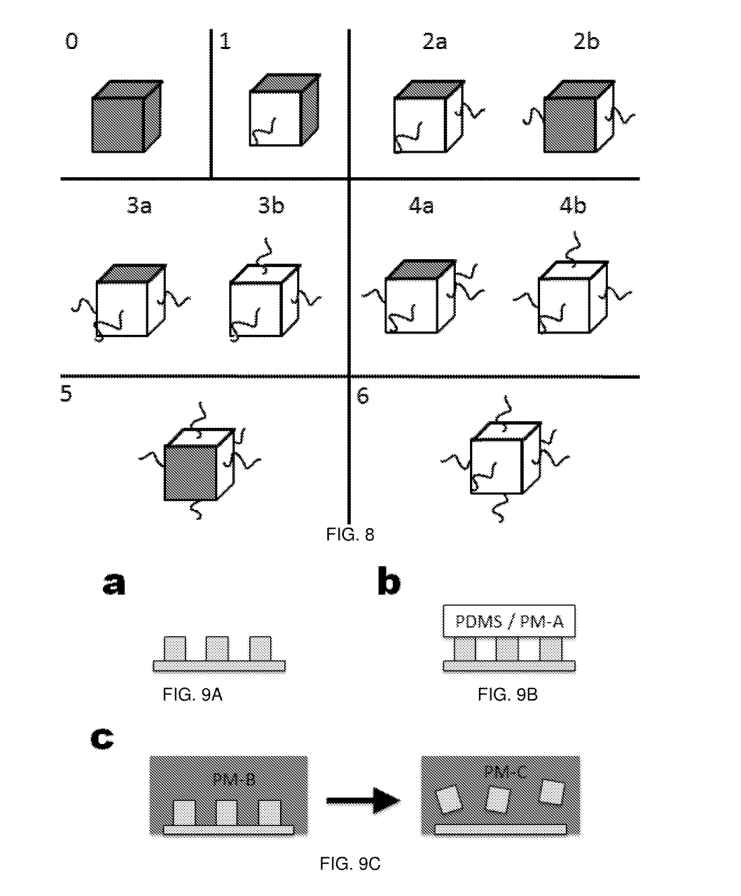

For purposes of illustration, consider the following example. A patched nanocube has been synthesized with six possible distinct species of ssDNA on each face. These distinct species shall be referred to as A, B, C, D, E, and F. After the reaction, some cubes feature one face-covered patch containing ssDNA of sequence A. Other cubes contain multiple patches of sequence A. Others still contain zero patches of sequence A. In FIG. 8, ten possible arrangements available for patch A are shown. They are labeled in this figure as 0, 1, 2a, 2b, 3a, 3b, 4a, 4b, 5 and 6, where the number refers to the number of faces containing sequence A and the letter distinguishes different morphologies with the same number of sequence A covered faces.

To isolate the products from the mixture, the nanocube mixture shown in FIG. 8 may be added to a solution or suspension containing a long molecular strand that binds to A. For example, one embodiment may involve adding 60-nucleotide long oligonucleotide A′ that is complementary to sequence A. Importantly, this oligonucleotide can be designed to hybridize only with faces on the nanocube that contain a patch populated by sequence A. Thus, in FIG. 8, nanocube faces coated with sequence A are shown with a long ligand extending from the surface. In practice, multiple long ligands may bind to various surfaces of various nanoparticles. The long ssDNA, A′, hybridizes only to patches containing sequence A. Those cubes with zero faces containing sequence A will not hybridize to the complementary strand A′. As such, they have the smallest effective radius. Those nanocubes containing exactly one face labeled with sequence A contain one face hybridized to the long complementary strand A′. These nanocubes have the second smallest effective radius. Accordingly, the different nanocubes can be separated from the mixture using procedures that are well known in the art. For example, one may separate the cubes using non-denaturing gel electrophoresis in an agarose gel, e.g., such that particles with the smallest effective radius move through the gel at the greatest rate. Each distinct arrangement of patches yields a different mobility in the gel. Upon physical extraction of the nanocubes from the gel matrix, the different nanocube with patches containing sequence A can be separated. This procedure may be repeated for each of the remaining DNA sequences. Up to six different sequences can be used to separate the nanocubes in FIG. 8, resulting in the purification and isolation of any one or all of the 6⁶=46,656 unique arrangements of patches on a DNA nanocube, a subset of which can then be selectively added to a mixture to create superstructures of a specific shape.

The likelihood of each arrangement of the patches shown in FIG. 8 is determined by kinetics, thermodynamics, and the stoichiometric ratio of strands during the synthesis. Not all arrangements of the face ligands are equally likely, nor are they equally useful. For instance, if one wishes to assemble linear wires of nanocubes, selectively binding patches may be required only at opposite ends of the nanocube. To obtain this particular pattern on the cube surface, in one embodiment, only one selectively binding patch along with a non-functional “junk” patch may be added. The “junk” patch can be any chemical bound to the nanocube surface that does not bind to patches on other particles.

It should be understood, however, that such methods are not limited to only cube-shaped nanoparticles, but may be extended to any other nanoparticle system. For example, in various embodiments, nucleic acid strands may be selectively bound to certain faces of a nanoparticle, e.g., suspected of containing desired patches, and the nanoparticles separated using gel electrophoresis or other techniques discussed herein. For instance, the nucleic acid strands may contain a portion substantially complementary to a nucleic acid believed to be present on a face of a nanoparticle, e.g., in a “patch” region. For example, the nucleic acid strand may contain at least 4, 5, 6, 7, 8, 9, or 10 consecutive sequences that are complementary to a sequence believed to be present on the face of a nanoparticle.

The nucleic acid strands may be of any suitable length. In some cases, the nucleic acid strands may be single-stranded, or have a sequence that does not have substantial self-complementarity (e.g., such that the sequence is unable to bind to itself to form a stable double-stranded structure; these complementary sequences typically are at least 6, 7, 8, or more consecutive nucleotides long). For example, the sequence may be at least 30, at least 50, at least 70, at least 100, at least 200, at least 300, at least 500, at least 700, or at least 1000 nucleotides long. However, it should be understood that in other embodiments, the nucleic acid strands may have one or more sequences that include substantial self-complementary regions.

In one aspect, various superstructures can be formed from nanoparticles such as those described herein. These may be formed using self-assembly or other techniques. For example, for nanoparticles such as nanocubes having faces featuring selectively binding patches may be combined, e.g., in solution or suspension, with other nanoparticles having complementary patches on one or more of their faces. In some cases, this process may be facilitated through stirring or other mechanical actions.

In one set of embodiments, the superstructure may comprise at least 2, at least 3, at least 5, at least 8, at least 10, at least 15, at least 20, at least 25, at least 30, at least 40, at least 50, at least 60, at least 70, at least 80, at least 90, at least 100, at least 150, at least 200, at least 300, at least 400, at least 500, at least 750, at least 1000, at least 3000, at least 5000, or at least 10,000 nanoparticles. In some cases, each of the nanoparticles have unique arrangements of patches. In other cases, however, some of the nanoparticles within the superstructure may be identical to each other.

Dimer aggregates may be formed as the complementary patches bind together. Larger aggregates comprised of more nanoparticles can also be formed in various embodiments, representing a general method for synthesizing arbitrarily shaped three-dimensional superstructures, regardless of how anisotropic or complex the target structure may be.

In some cases, the nanoparticles may be considered to represent a “pixel” (e.g. a nanocube pixel) within a larger superstructure, in two or three dimensions. The patches may be selected so as to determine where each “pixel” will appear within the superstructure. By controlling the location of patches on individual nanoparticles, complex superstructures may be obtained with almost any suitable shape. In some cases, the synthesis may involve only one type of building block (e.g., only one type of nanoparticle), which may reduce the complexity of the assembly process, while simultaneously expanding the complexity of the superstructures that can be built. As such, this method reduces the number of synthetic techniques one needs to assemble a variety of different shapes, and could be adopted as a standardized technique to assemble large classes of superstructures. However, it should be understood that in other embodiments, more than one type of nanoparticle may be present, e.g., having different shapes, sizes, materials, etc., as discussed herein.

For illustrative purposes, imagine synthesizing a set of cubes whose faces each contain a patch of some predetermined sequence of ssDNA. Each face is covered by one ssDNA sequence. Now imagine a second set of cubes synthesized in the same manner, except that one face contains a DNA sequence complementary to another sequence on the first set of nanocubes. Since single-stranded DNA will only bind with its complementary strand, these cubes bind together at complementary faces. This allows control of which faces bind together to form dimers. To form superstructures, many nanocubes may be synthesized, some or all of which may have various unique DNA patches in order to from the superstructure.

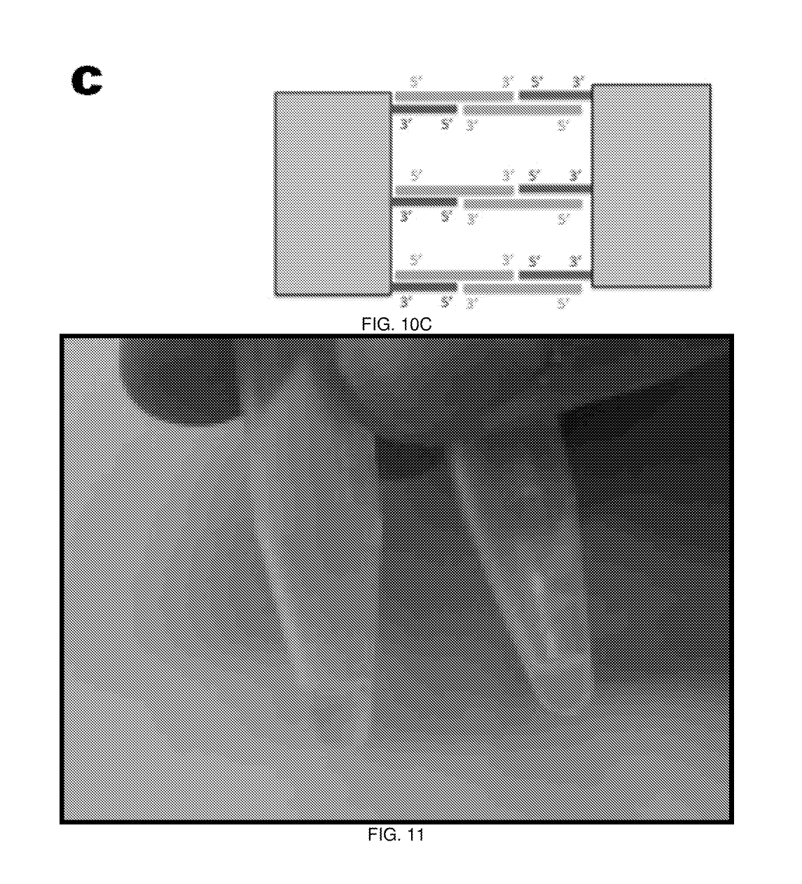

In various embodiments, the nanoparticles can be combined into larger structures of arbitrary shapes. Many methods of combination are possible. FIG. 10 depicts several example mechanisms of connecting ssDNA-coated nanoparticles (e.g., nanocubes), including direct connections (top), single-strand linkers (middle), and double-strand linkers (bottom). (It should be understood that although DNA is shown here, this is by way of example only, and in other cases, other nucleic acids such as RNA, PNA, XNA, or combinations of nucleic acids may be used, e.g., having binding configurations such as those shown in FIG. 10) In addition, in some embodiments, combinations of these and/or other approaches may be used. This illustration only shows one face of each of the nanoparticle for clarity, and is not representative of the expected number of DNA ligands on the surface or of the shape of the nanoparticle. FIG. 11 shows a suspension of DNA-nanocubes with the linker added at low (left tube) and high (right tube) temperature as a specific example. At lower temperature, the nanocubes aggregate and appear red. At high temperature, the aggregates melt and appear blue. Accordingly, the binding of nanocubes can be controlled.

In one embodiment, nanoparticles are directly connected to each other, e.g., in a face-to-face orientation, to form a superstructure. It should be understood that the orientation may be exact, or in some cases, the alignment of nanoparticles may be off-center. As a specific non-limiting example, DNA ligands covering a face of one nanoparticle may hybridize to DNA ligands on the face of another nanoparticle. By preparing the nanoparticle faces with known DNA sequences in advance, as discussed herein, and then combining the nanoparticle in solution or suspension, aggregates may form as the DNA-coated faces bind to other faces containing the complementary strand. If the connections are unique, then only a specific superstructure may form, e.g., one that is programmable or predetermined.

However, while linker DNA is not necessarily used in all embodiments, linker DNA can be used in some cases. For example, when forming large structures, the kinetics may result in higher yields if the hybridization reactions proceed in a certain order. Adding linker strands in progression, e.g., to the solution or suspension containing nanoparticles, may control the order in which nanoparticles bind together to form the larger structures.

Yet another embodiment uses the addition of an ssDNA (or other suitable nucleic acid) as a linker to initialize the hybridization of multiple nanoparticles. To build structures from the nanoparticle, one may combine the nanoparticles to be linked in solution or suspension along with appropriate ssDNA linker strands. The addition of a linker may allow, in some cases, the order in which nanoparticles bind to each other to be specified. In some cases, for example, this may increase the yield of the superstructures by avoiding kinetic traps, e.g., where the correct superstructure is not able to be formed.

For example, as shown in FIGS. 10B and 10C, in some cases, in some cases, two faces may have nucleic acids which do not directly specifically bind to each other, but which each may bind to one or more linker strands which, in turn, allow specific binding to effectively occur between the faces of the nanoparticles. It should be understood that the linker strands may be any suitable nucleic acid, such as those discussed herein, and may independently be the same or different from the strands on the faces of the nanoparticles.

In some embodiments, self-assembly can be characterized by UV-Vis spectroscopy or other suitable techniques. The aggregation of the nanoparticles may be observed as a shift in the surface plasmon resonance, which changes the color of the solution or suspension, e.g., from red to blue as shown in the example of FIG. 11.

Still another example embodiment of binding DNA utilizes double stranded DNA as a “bolt” to connect nanoparticles. The double stranded “bolt” has two sticky single-stranded ends that are complementary to the ssDNA on the faces that will be bound. When added to solution or suspension with the nanoparticles, the bolt can bind to any cube faces whose ssDNA contain complimentary sequences to the sticky ends of the bolt. This allows nanoparticles having nucleic acids on faces that are modified on one end of the nucleic acid (e.g. the thiolated 5′ end as shown in FIG. 10A). In some cases, this may allow the order in which nanoparticles bind to be controlled, e.g., through addition of the bolt linker at suitable times.

Certain aspects of the present invention are generally directed to superstructures that are formed as discussed herein. In some cases, for example, suitable nanoparticles may be induced to assemble together to form a superstructure, for example, spontaneously (e.g., self-assembly), and/or through the addition of other agents, such as linker strands or bolts, to cause assembly to occur. In some cases, a single superstructure is assembled from nanoparticles; in other cases, however, more than one such superstructure may be assembled, e.g., when using multiple substantially identical nanoparticles. Thus, in some embodiments, a plurality of superstructures are formed. In some cases, at least 20%, at least 30%, at least 40%, at least 50%, at least 60%, at least 70%, at least 80%, at least 90%, at least 95%, or substantially all of the superstructures that are formed may share essentially identical configurations of nanoparticles forming those superstructures. In one set of embodiments, the superstructures are formed in a solution or suspension comprising nanoparticles. In some cases, the superstructures that are formed are solid or stably formed from nanoparticles, e.g., the superstructure has a well-defined shape or structure under ambient conditions (e.g., at room temperature and pressure). In some embodiments, the superstructure may be stable or have a solid form even when contained within solution or suspension, e.g., such that the superstructure does not typically dissociate or “fall apart” when left undisturbed under room temperature and ambient pressure, even in the presence of normal fluidic flow within the solution or suspension. The shape of the superstructure can be programmed or predetermined in certain instances, e.g., as discussed herein.

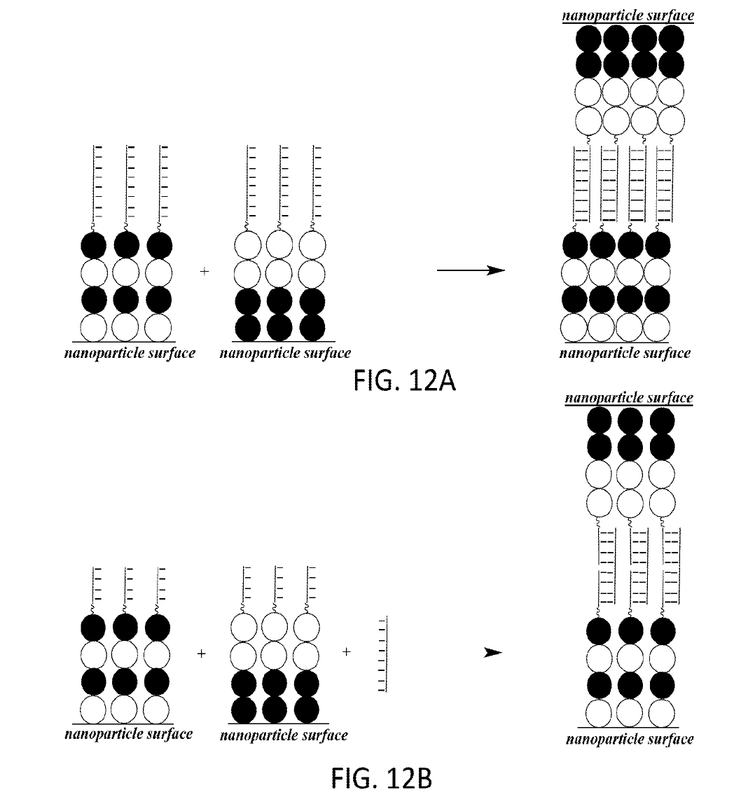

For example, in FIG. 12, multiple nanoparticles are assembled using nitrogenous bases interacting via Watson-Crick base pairing. FIG. 12A illustrates nanoparticles assembled by direct interaction of the polymers on the surface through complementary nitrogenous base sequences. In FIG. 12B, polymers with non-complementary nitrogenous base sequences can be assembled by addition of an oligonucleotide linker that contains complementary sequences to each of the polymers.

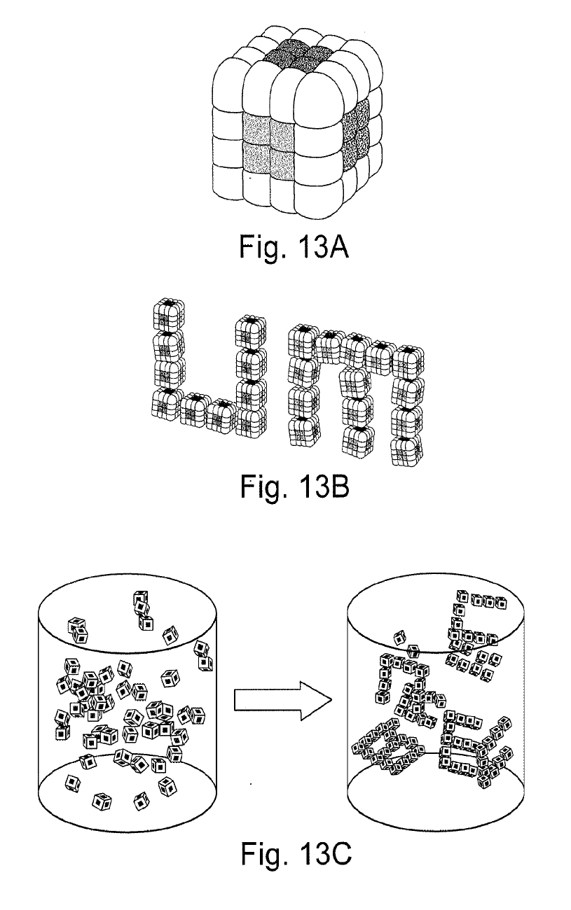

FIG. 13 depicts a schematic of an exemplary embodiment of a patched nanocube assembly, as a non-limiting example. In FIG. 13A, a patched nanocube is illustrated with selectively binding patches isolated on each face. Each patch species is represented in the figure by a different shade of gray centered on the face of the nanocube. In FIG. 13B, by arranging the patches on the surface of multiple building blocks, one can design arbitrarily shaped target structures because the particles will only bind in specific ways. By simply arranging complimentary binding patches on the adjacent faces in the structure, one can preprogram the assembly instructions directly on the particle surface. In FIG. 13C, a mixture of patched nanocubes in solution or suspension self-assembles into predesigned structures. Self-assembly may occur through selective binding interactions that cause the facetted particles to bind face-to-face. By isolating selectively binding ligands on different faces, one can control the assembly, as specific faces will only bind to another face if it contains a complementary ligand.

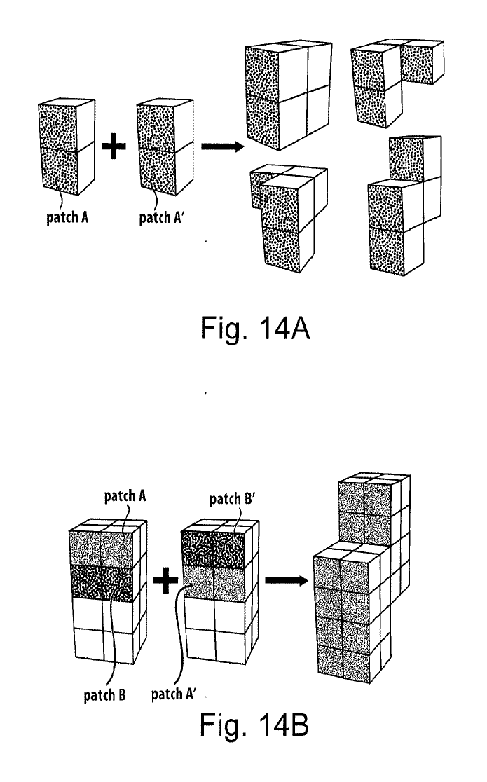

Consider, as an illustrative non-limiting example, eight nanocubes fused into a larger cubical building block. These building blocks, since they are made of smaller cubes that have been patched in separate syntheses, may contain multiple selectively binding patches on each face. Consider two such building blocks, where the first contains a face that has been patched with species A and B, and the second has been patched with species A′ and B′, as illustrated in the dimers of FIG. 14B. Here, the dimers have two cubical building blocks, each of which is composed of eight smaller cubes. Suppose that patches A′ and B′ are complimentary to A and B, respectively. When combined, these cubes will form a direction specific bond, as illustrated in the right hand side of FIG. 14B. By orienting the patches in other ways, one can obtain any of the four possible binding orientations illustrated on the right hand side if FIG. 14A.

Such superstructures can be used in a wide variety of applications, according to various aspects of the invention. Non-limiting examples of applications using these systems are discussed herein, and include, for instance, including colloidal crystal synthesis, emulsions, electronic ink, novel optical properties, sensors, rheological probes, shape shifters, and self-healing materials. Other examples include, but are not limited to, the following: biomedical devices (e.g. drug delivery, artificial enzymes, and the like); catalysis for organic/chemical syntheses used in medicine, agriculture, energy technologies, and the like; biosensors and diagnostics; photonics crystals; Information technology and nanoelectronics including nanowires, transistors, 2D/3D circuit arrays, computer memory and information storage, and quantum bits for quantum computers; nanorobotics; materials (e.g. polymers, fibers, films, ceramics, thermoelectrics, piezoelectrics, and the like); purification and separation; clean energy technologies including energy storage (i.e. batteries) and energy harvesting (e.g. artificial photosynthesis and catalytic synthesis of biofuels); and energy transfer to nanoscale systems (e.g. antennas). Some specific representative examples of these are discussed in the examples below.

U.S. Provisional Patent Application Ser. No. 62/195,175, filed Jul. 21, 2015 is hereby incorporated herein by reference in its entirety.

Although the disclosure has been described with reference to preferred embodiments, persons skilled in the art will recognize that changes may be made in form and detail without departing from the spirit and scope of the disclosed apparatus, systems and methods. The following examples are intended to illustrate certain embodiments of the present invention, but do not exemplify the full scope of the invention.

### Example 1

In an exemplary embodiment, nanowires may be constructed. In such an embodiment, nanocubes are assembled with selectively binding patches on opposite faces. For purposes of illustration, the patches may be comprised of ssDNA. In this embodiment, the faces of nanocubes three species of patches: (1) ssDNA of sequence A on the top of the cube, (2) ssDNA of sequence B on the top of the cube, and (3) ssDNA of sequence C on the four remaining faces around the circumference of the cube. A may be bonded to B using, for example, a single stranded linker method. When added to a solution or suspension of the nanocubes, the complementary “sticky” ends of the linker hybridize to DNA patches A and B, linking adjacent cubes in the nanowire. A cube is a repeating subunit, which in essence, acts like a repeating monomer in a nanoparticle polymer.

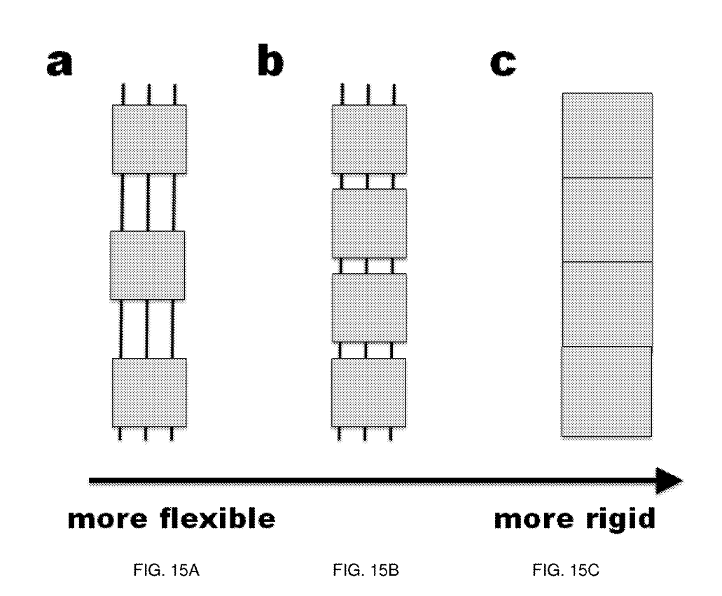

The linker DNA strand may be synthesized with variable length and with different proportions of binding and non-binding regions. The non-binding regions remain single stranded. Depending on solvent conditions, ssDNA regions can be many times more flexible than double stranded regions. A bolt containing a large region of single stranded DNA will generate a very flexible nanocube wire (FIG. 15A). By reducing the length of the single stranded region, the nanocubes wire will become less flexible and more rigid (FIG. 15B). By eliminating the single stranded region entirely, the nanocube wire becomes an inflexible rigid beam (FIG. 15C). Note that these wires leave open a third selectively binding patch C, to which can be added any of a variety of possible functionalities.

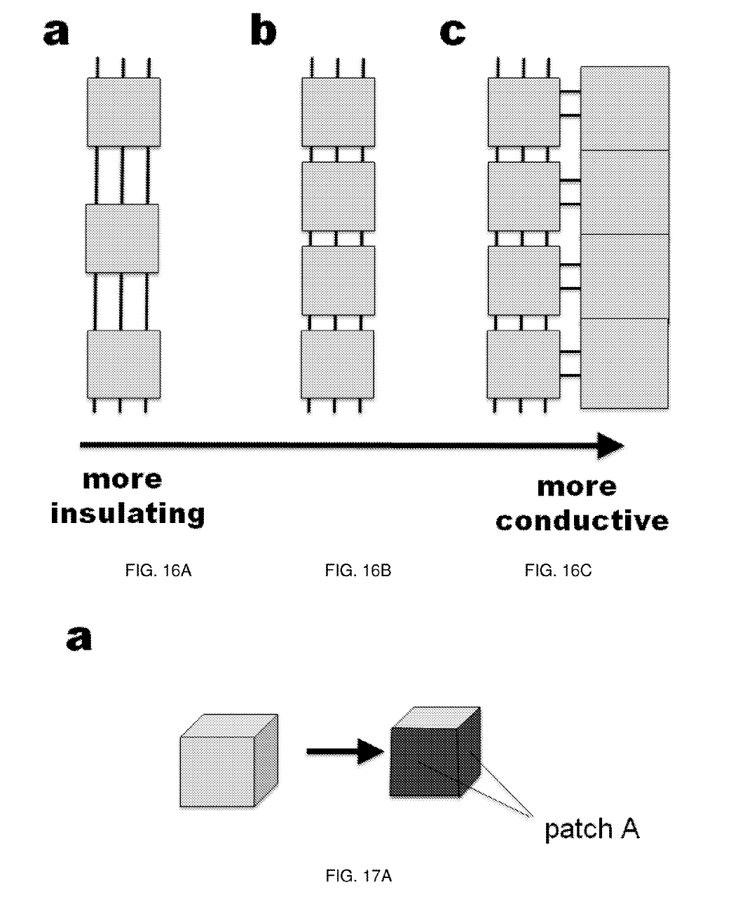

Various embodiments of these nanowires can be used to adjust its conductivity. Depending on the humidity, temperature, and other environmental conditions, DNA has been shown to conduct electricity over short distances, but not long distances. In contrast, metallic cubes are conductive in any appreciable amount. By varying the size of the cubes along with the binding DNA length, the nanowires can be tuned to have any value of conductivity between pure DNA and pure gold. Metallic nanocubes connected by long DNA strands will be predominantly insulating, because DNA can conduct charges only over short distances (FIG. 16A). By decreasing DNA strand length, the conductivity of the wires increases, and current can travel up and down the length of the wire, as illustrated in FIG. 16B. The DNA between the conductive nanocubes can be removed completely if they are held in place by scaffolding provided by another set of DNA coated nanocubes, as illustrated in FIG. 16C. Under this arrangement, the conductivity will approach that of bulk gold.

### Example 2

In another exemplary embodiment, sheets may be created from the nanocubes. Consider again, as an example, nanocubes with three patches: patch A on the top face, patch B on the bottom face, and patch C on the four faces around the circumference (FIG. 17A). In this embodiment, patch C is made to be complimentary to itself. When combined in solution or suspension, these nanocubes bond face-to-face to assemble sheets (FIG. 17B). A cube is a repeating subunit, which acts like a repeating monomer in a nanoparticle polymer. As with nanowires, unbound patches, in this case patches A and B, can be used to add functionality to the sheets.

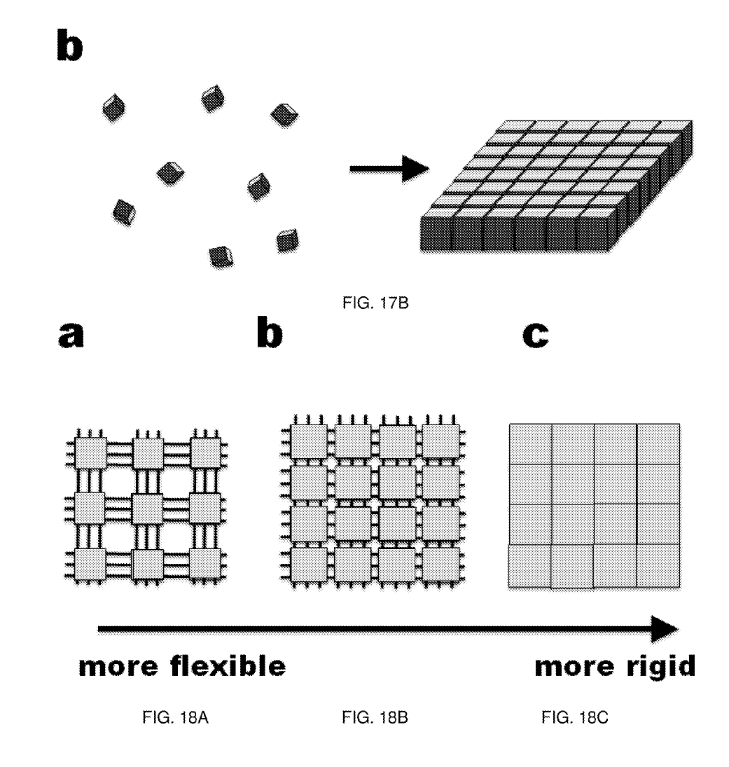

Adjusting the thickness of the cubes during their synthesis can control the thickness of the sheets. The flexibility and conductivity of the sheets can be tuned in a manner similar to that of the nanowires. By way of illustration, nanocubes can be synthesized with selectively binding DNA patches around their circumference, as illustrated in FIG. 18. A third DNA strand (straight black line between the cubes) is added to solution or suspension (FIG. 18A). This DNA contains sticky ends that are complementary to the DNA patches on the surface of the nanocubes. A region of single stranded DNA separates these sticky ends. A large region of single stranded DNA generates a more flexible sheet. By reducing the length single stranded DNA in the bolt, the self-assembled nanocube sheet will become less flexible and more rigid (FIG. 18B). By eliminating the single stranded region entirely so that the DNA on adjacent cubes bind directly to each other, the nanocube sheet becomes an inflexible rigid plane (FIG. 18C).

### Example 3

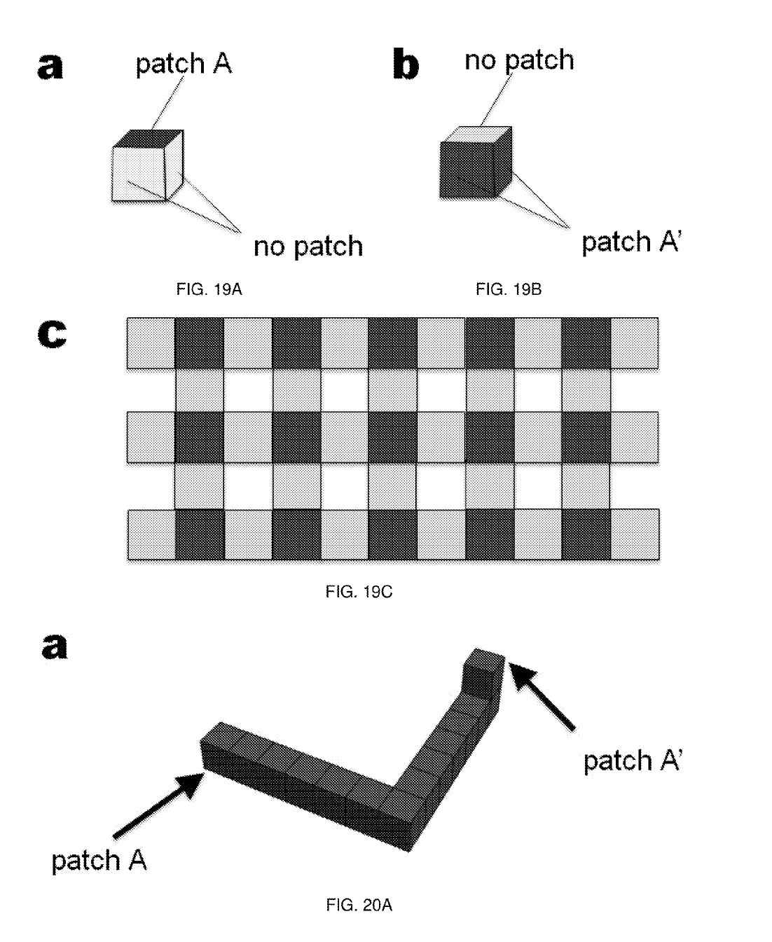

In another exemplary embodiment, porous sheets are produced. A set of nanocubes is synthesized with a DNA patch A on two opposite faces of the cube, while the remaining four patches contain unspecific binding patches (FIG. 19A). A second set of cubes is synthesized containing the complementary patch A′ on the four faces around its circumference, while the top and bottom contain unspecified binding patches (FIG. 19B). By combining these two nanocubes, one obtains porous sheets where each pore (white square) is surrounded by four cubes of the first type of cube (light gray) and four of the second type (dark gray). The unspecified binding patched may again be used to add functionality to the sheet. Possible applications of this embodiment include catalytic reactor surfaces, porous filtration systems, and nanopores.

### Example 4

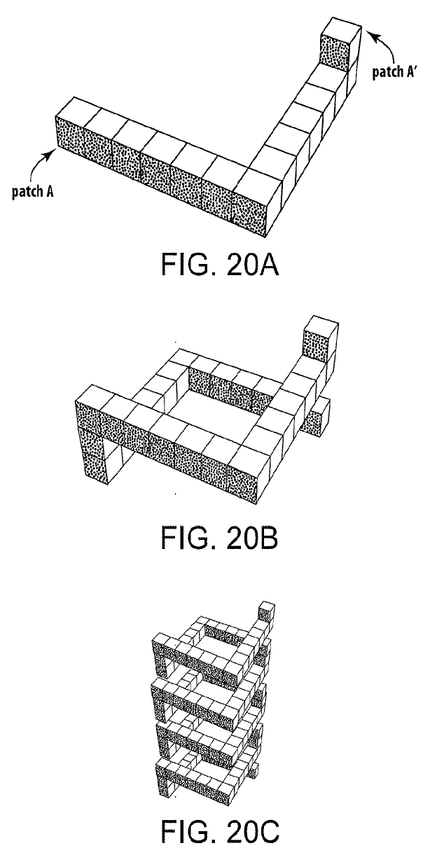

Yet another exemplary embodiment is a helix. In this embodiment, individual patched nanocubes are first assembled in an L-shape as shown in FIG. 20. This can be accomplished straightforwardly by combining (1) multiple species of cubes containing two binding patches on opposite faces with (2) cubes containing two binding patches on adjacent faces. Here, the latter makes use of the directionally binding patches described earlier. The L-shape denotes the repeated monomeric unit inside the helix. Patches A and A′ denote the complementary selectively binding directional patches where additional monomeric units will be added (FIG. 20A). FIG. 20B illustrates how a second monomeric L-shaped unit's patch A′ bonds to patch A underneath the original L-shape from FIG. 20A. Repeated addition of monomeric L-shaped units generates a helix of nanocubes (FIG. 20C).

Exemplary applications of helical structures include solenoids, inductors, transducers, transformers, electrical relays, artificial flagella, and electromagnets. For instance, a helical coil may be connected to some nanoscale device that requires electrical power. By placing the nanoscale device inside a varying magnetic field (e.g., one produced by a pulsed NMR) and delivering a short burst of magnetic flux through the coil, Faraday's law dictates that an electric motive force (emf) will be produced inside the coil. This emf produces an electric current in the device that provides electrical power.

### Example 5

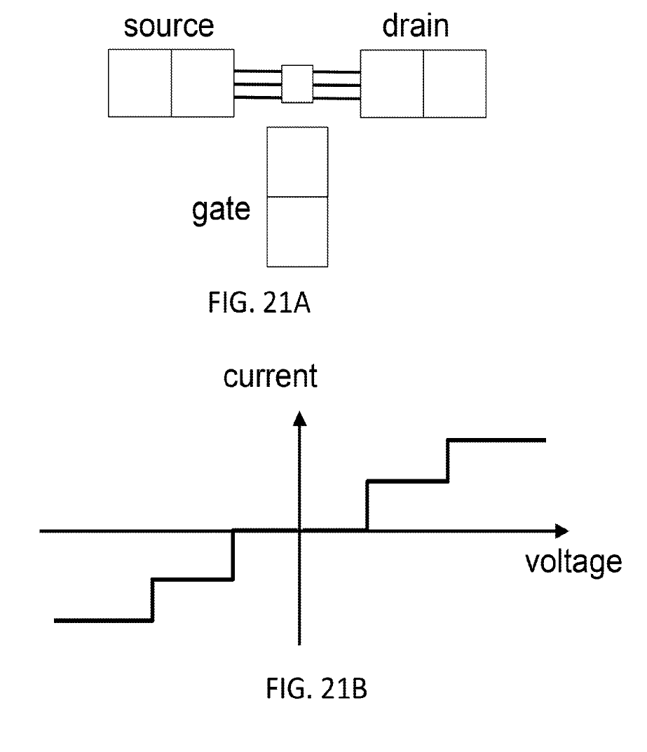

In yet another exemplary embodiment, the patched nanocubes can be assembled into transistors and logic gates. In this embodiment, nanocubes are self-assembled into a source, drain, and gate for a field-effect transistor as illustrated in FIG. 21A. A scaffold (not shown) is used to position the source, drain, and gate relative to each other. In some embodiments, the scaffold may itself be composed of nanocubes. The source, drain, and gate are the connected to other circuitry by nanowires (not shown). A smaller nanoparticle quantum dot is connected to both the source and drain by a linker molecule.

Operation of the transistor is familiar to anyone immersed in the art. Briefly, a voltage difference V_{ds} is applied across the source and drain. When a biasing voltage V_{g} is applied to the gate, the current between the source and the train can be turned on/off as illustrated in the chart of FIG. 21B. For instance, when the gate is given a negative biasing voltage, electrons cannot pass to the quantum dot because of the coulombic repulsion between like charges. If made small enough (˜5 nm) the quantum dot operates as a single electron transistor, in which individual electrons tunnel on and off the quantum dot island. The charge-charge repulsion results in the well-known coulomb blockade. The creation of modular transistors permits the creation of more complex logic gates (e.g. AND, OR, XOR, NAND, NOR, etc.) which can be combined to create computing devices.

### Example 6

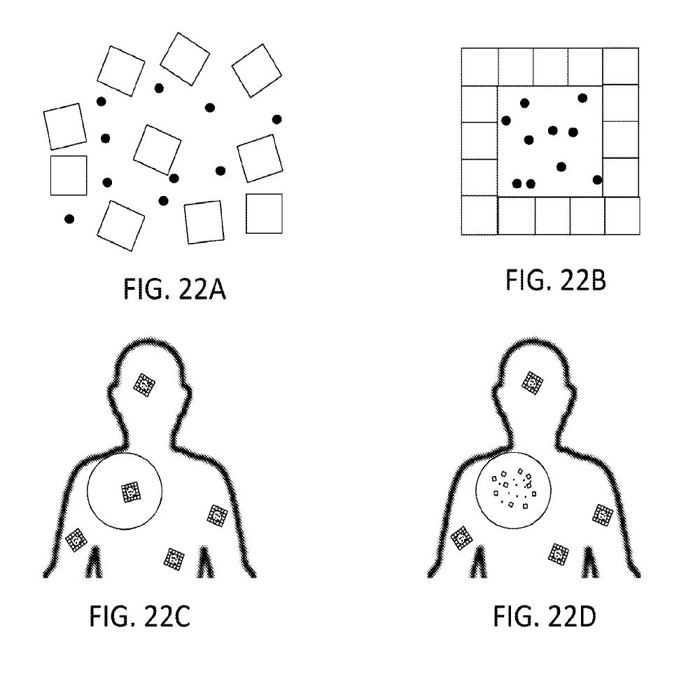

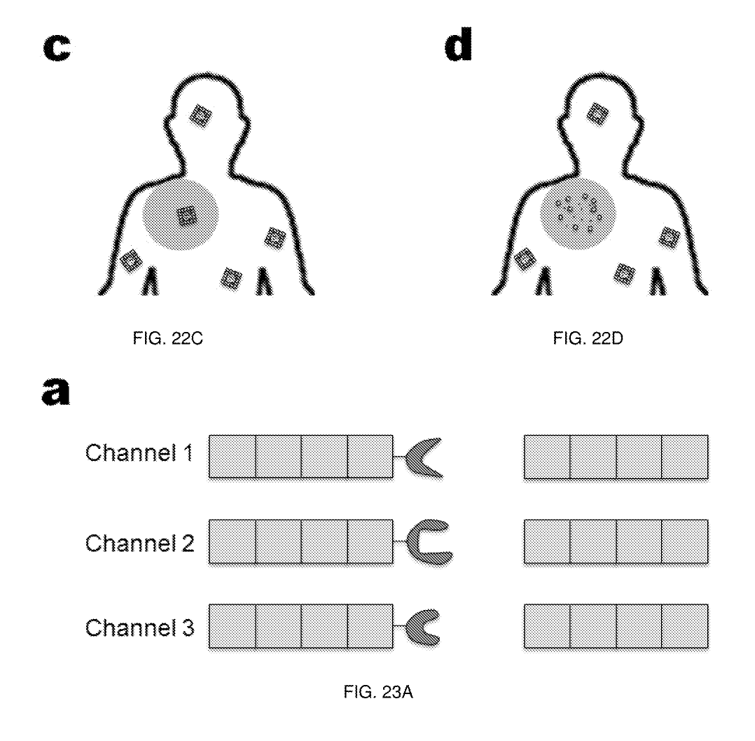

In a further exemplary embodiment, a drug delivery cage can be constructed. In one such embodiment, the cage features a hollow core, so as to be capable of housing a medicine. By controlling the location of selectively binding patches on individual cubes, the superstructure can be designed such that it can be almost any shape. In one embodiment, the cubes comprising the box are added to a solution or suspension containing drug molecules as illustrated in FIG. 22A. As the box self-assembles, it traps some of the drug molecules inside (FIG. 22B). The patient ingests the boxes. The boxes diffuse through the patient's body (FIG. 22C). Some of the boxes enter the afflicted area (gray circle), while the rest disperse throughout the patient's body. The box is then selectively opened via a variety of potential methods. By way of example, applying a varying magnetic field to the afflicted region only will inductively heat the boxes in that region by the same process that metals are inductively heated in induction welding. This heat raises the temperature locally around the boxes in the afflicted region while leaving the rest of the body unaffected. The temperature increase melts the patch bonds holding the box together and the nanocubes separate from each other. In this process the drug is released only in the affected regions while leaving the healthy regions free from side effects of the drug.

### Example 7

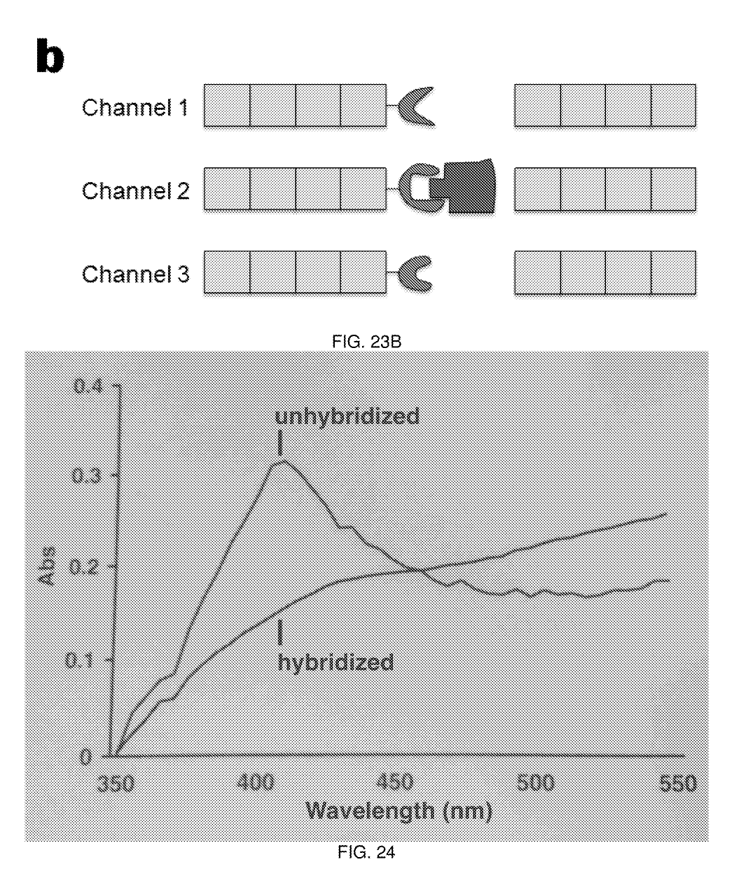

Yet another embodiment involves the detection of different chemicals species. In this embodiment, several nanocube wires are assembled in different channels as illustrated in FIG. 23. Each channel contains a gap that is large enough to fit a molecule of interest. The edges of the gap are coated with a receptor for the molecule of interest. The receptor has been added to the cube by chemically bonding it to a complement for the patch of that cube. See FIG. 23A. When the molecule is present (e.g., FIG. 23B), it binds to a receptor. By binding, the electrical properties (e.g. capacitance, conductivity, etc.) of channel 2 are altered, which is used to determine the presence of the molecule.

### Example 8

This example illustrates synthesis of a silver nanoparticle with an average edge length of 45 nm. 6 mL of ethylene glycol was added to a 25 mL round bottom flask. Ethylene glycol was heated to 160° C. in an oil bath while stirring with a Teflon-coated magnetic stirring bar for 1 hour. 0.008 mL of a freshly prepared 3 mM NaHS solution in ethylene glycol was added to the reaction. An ethylene glycol solution of 20 mg/mL powder polyvinylpyrrolidone (average molecular weight ˜55,000) was added to the reaction mixture, followed quickly by the addition of 0.5 mL of a 50 mg/mL AgNO₃ solution in ethylene glycol. Formation of the nanocubes can be followed by observing the visible color changes significant of different nanoparticle morphologies that form and decompose as the reaction progresses. Small, silver nanoparticles were observed initially as a pale yellow color that changes to the opaque green ochre indicative of the formation of 45 nm edge length silver nanocubes after 30-45 min. The reaction was quenched in a room temperature water bath. The nanocubes were washed once with acetone and three times with water. Nanocubes were stored at 4° C. in water.

### Example 9

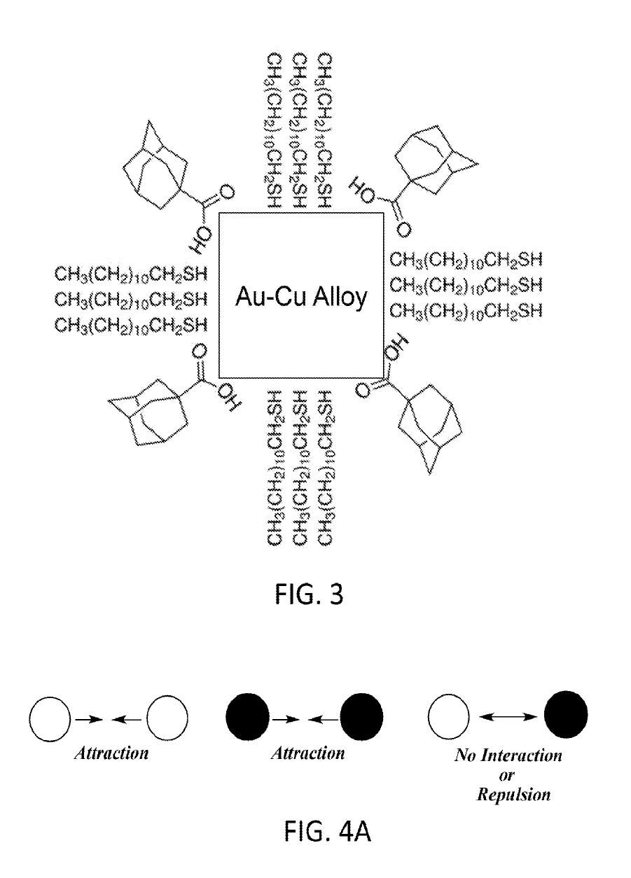

This example illustrates synthesis of a gold-copper nanoparticle. Copper (II) acetylacetonate and gold chloride (HAuCl₄) are combined with 1,2-hexadecanediol (HDD) in a diphenyl ether solvent. HDD, a mild reducing agent, simultaneously reduces both the copper and gold ions, which aggregate together as a single Cu—Au alloy. Alkanethiol, 1-dodecanethiol (DDT) is then added as a capping agent to inhibit the growth of the Cu—Au crystal at nanoscale sizes. FIG. 3 is an illustration of the 1-dodecanethiol (DDT) and 1-adamantane carboxylic acid (′) capping agents positioned on a Au—Cu nanocube surface.

By itself, DDT does not break the symmetry on the surface, which results in spherical rather than cubical nanoparticles. A second, bulky capping agent, 1-adamantane carboxylic acid (ACA), is added to form cubic morphology. ACA migrates to regions of the nanoparticle surface with greater free volume, enabling edges and facets form. The nanocubes may then be isolated via centrifugation. The supernatant, which contains the various byproducts of the reaction, is decanted to waste. The nanocubes are then washed and resuspended in toluene.

As noted above, monolayers of functionalized alkanethiol ligands on the surface of gold nanoparticles (AuNP) can be exchanged via place-exchange reactions, such as:

(R′—SH)+(R—S)—AuNP→(R—SH)+(R′—S)—AuNP (Eq. 1)

In this generalized reaction, thiols functionalized with group R are exchanged for thiols functionalized with group R′. To ensure exchange, R′—SH can be added in excess to the reaction.

Using this ligand exchange methodology, an alkanethiol capping group such as DDT may be exchanged with amphipathic 6-mercaptohexanoic acid (MHA) to facilitate dissolution in an aqueous solvent. Excess MHA is added to the nanoparticles in toluene and heated. The resulting MHA capped nanoparticles are purified by centrifugation and washed before resuspension in aqueous buffer.

### Example 10

This example illustrates application of DNA a silver nanocube surface by ligand exchange in aqueous solution. HPLC purified DNA oligonucleotides were synthesized with a terminal monothiol adjacent in the sequence to a spacer (Integrated DNA Technologies). The monothiol was positioned on either terminal end of the oligonucleotide. Both oligonucleotides contain a 15 base complementary sequence. The disulfide was deprotected by incubating DNA in a buffered aqueous solution (10 mM phosphate, pH 7.5) with 100-fold molar excess Tris (2-carboxyethyl) phosphine for two hours at room temperature.

3.5 μM solution of the deprotected, single-stranded DNA is incubated with 0.1 nM nanocubes suspended in buffered aqueous solution (10 mM phosphate, pH 7.5, 0.15 M NaCl) for 12 hours. Excess free DNA is removed from the solution by washing the nanocubes three times with the buffered aqueous solution (10 mM phosphate, pH 7.5, 0.15 M NaCl).

### Example 11

This example illustrates application of DNA a silver nanocube surface by contact stamping. A monolayer of nanocubes was created by applying 0.2 mL of a 0.1 mM solution of nanocubes suspended in ethanol to a freshly cleaved 1 cm mica disc (Ted Pella, Inc.). The ethanol was evaporated with a stream of nitrogen. 0.02 mL of a 0.01 mM solution of deprotected DNA oligonucleotides containing a terminal monothiol in a phosphate buffer (300 mM phosphate, pH 9.0) was applied to the flat surface of a polydimethylsiloxane (PDMS) stamp (Sylgard 184, Dow Corning). The DNA solution was allowed to incubate on stamp surface for 0.5 min before being dried under a stream of nitrogen. The DNA coated face of the stamp was applied manually to the monolayer of nanocubes on the mica disc and held in place for 2 min. The mica disc was rinsed with aqueous buffer (300 mM phosphate, pH 9.0). The nanocubes were removed from the mica surface by sonicating the disc in aqueous buffer (300 mM phosphate, pH 9.0).

### Example 12

This example illustrates binding of nanocubes to form a superstructure, in accordance with some embodiments. Nanocubes with complementary binding faces were assembled together by combining functionalized nanoparticles in an aqueous solution buffered to pH 7.4 with 10 mM sodium phosphate and an NaCl concentration of 1.0 M. The final concentration of nanocubes was 0.1 nM. Incubating at 23° C. for 5 hours allowed for hybridization of complementary DNA sequences to occur. Assembly of the complementary binding nanocubes was observed by monitoring the decrease of the maximally absorbing visible wavelength (Agilent Carey 60 UV-Vis). Unhybridized silver nanocubes with an edge length of 45 nm display an intense absorbance of visible light around 420 nm wavelength due to surface plasmon resonance. Hybridization of the nanocubes caused the maximally absorbing wavelength of the plasmon resonance to shift to higher wavelengths, resulting in a significant change in the absorbance spectrum do to a decrease in the extinction coefficient at 420 nm (FIG. 24).

The proportion of successfully assembled structures could be modulated by the reaction conditions. For example, ionic strength of the aqueous solution could be controlled by NaCl or MgCl₂ concentration. The temperature of the aqueous solution could be precisely controlled, e.g., using a dry heating block. The concentration of components in the aqueous solution could be controlled by volume and initial concentration of each component added to the solution, in addition to the rate of addition of the components.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a superstructure comprising at least three nanoparticles, joined in face-to-face contact to form the superstructure, each face-to-face contact of the superstructure being defined by a binding interaction between the respective contacting nanoparticles, wherein each of the binding interaction within the superstructure of nanoparticles comprises no more than 10% of the total binding interactions within the superstructure of nanoparticles.

2. The composition of claim 1, wherein each binding interaction within the superstructure of nanoparticles is unique.

3. The composition of any one of claim 1 or 2, wherein at least some of the binding interactions are nucleic acid interactions.

4. The composition of any one of claims 1-3, wherein at least some of the binding interactions are hydrogen bond interactions.

5. The composition of any one of claims 1-4, wherein at least some of the binding interactions are covalent couplings.

6. The composition of any one of claims 1-5, wherein at least some of the binding interactions are hydrophobic interactions.

7. The composition of any one of claims 1-6, wherein at least some of the nanoparticles are polyhedral nanoparticles.

8. The composition of any one of claims 1-7, wherein at least some of the nanoparticles are nanocubes.

9. The composition of any one of claims 1-8, wherein each of the nanoparticles within the superstructure are nanocubes.

10. The composition of any one of claims 1-9, wherein each of the nanoparticles within the superstructure are nanocubes.

11. The composition of any one of claims 1-10, wherein at least some of the nanoparticles have a largest internal dimension of less than about 1 micrometer.

12. The composition of any one of claims 1-11, wherein at least some the nanoparticles comprise a metal.

13. The composition of any one of claims 1-12, wherein at least some the nanoparticles comprise gold.

14. The composition of any one of claims 1-13, wherein at least some the nanoparticles comprise copper.

15. The composition of any one of claims 1-14, wherein at least some the nanoparticles comprise a semiconductor.

16. The composition of any one of claims 1-15, wherein at least some the nanoparticles comprise silicon.

17. The composition of any one of claims 1-16, wherein the superstructure comprises at least ten nanoparticles.

18. The composition of any one of claims 1-17, wherein the superstructure comprises at least 100 nanoparticles.

19. The composition of any one of claims 1-18, wherein each of the nanoparticles within the superstructure comprises a unique arrangement of patches.

20. The composition of any one of claims 1-19, wherein no more than 50% of the nanoparticles forming the stable superstructure are identical.

21. The composition of any one of claims 1-20, wherein the superstructure is contained within a suspension.

22. The composition of any one of claims 1-21, wherein the superstructure is stable.

23. A composition, comprising:
a superstructure comprising at least three nanoparticles bonded together via specific binding interactions, wherein each of the binding interactions within the superstructure of nanoparticles comprises no more than 10% of the total binding interactions within the superstructure of nanoparticles.

24. The composition of claim 23, wherein each binding interaction between the nanoparticles forming the superstructure is unique within the superstructure.

25. The composition of any one of claim 23 or 24, wherein at least some of the binding interactions are nucleic acid interactions.

26. The composition of any one of claims 23-25, wherein at least some of the nanoparticles are nanocubes.

27. The composition of any one of claims 23-26, wherein the superstructure comprises at least ten nanoparticles.

28. The composition of any one of claims 23-27, wherein no more than 50% of the nanoparticles forming the superstructure are identical.

29. The composition of any one of claims 23-28, wherein the superstructure is contained within a suspension.

30. A composition, comprising:
a stable superstructure comprising at least three nanoparticles, wherein at least two of the nanoparticles are not in contact with each other within the superstructure.

31. The composition of claim 30, wherein at least some of the nanoparticles are polyhedral nanoparticles.

32. The composition of claim 31, wherein the superstructure is formed from the polyhedral nanoparticles positioned in face-to-face contact.

33. The composition of any one of claims 30-32, wherein the stable superstructure is contained within a suspension.

34. The composition of any one of claims 30-33, wherein each of the nanoparticles within the stable superstructure comprises a unique arrangement of patches.

35. The composition of any one of claims 30-34, wherein no more than 50% of the nanoparticles forming the stable superstructure are identical.

36. The composition of any one of claims 30-35, wherein the nanoparticles forming the stable superstructure are in face-to-face-contact.

37. The composition of any one of claims 30-36, wherein at least some of the nanoparticles are nanocubes.

38. The composition of any one of claims 30-37, wherein the superstructure comprises at least ten nanoparticles.

39. The composition of any one of claims 30-38, wherein no more than 50% of the nanoparticles forming the superstructure are identical.

40. The composition of any one of claims 30-39, wherein the superstructure is contained within a suspension.

41. A composition, comprising:
a stable superstructure formed from a plurality of nanoparticles, wherein no more than 50% of the nanoparticles forming the superstructure are identical.

42. The composition of claim 41, wherein the nanoparticles forming the stable superstructure are in face-to-face-contact.

43. The composition of any one of claim 41 or 42, wherein at least some of the nanoparticles are nanocubes.

44. The composition of any one of claims 41-43, wherein the superstructure comprises at least ten nanoparticles.

45. The composition of any one of claims 40-44, wherein the superstructure is contained within a suspension.

46. A composition, comprising:
a plurality of superstructures formed from nanoparticles bound together by noncovalent interactions, wherein at least 50% of the superstructures comprise at least three nanoparticles and are indistinguishable.

47. The composition of claim 46, wherein the nanoparticles forming the stable superstructure are in face-to-face-contact.

48. The composition of any one of claim 46 or 47, wherein at least some of the nanoparticles are nanocubes.

49. The composition of any one of claims 46-48, wherein the superstructure comprises at least ten nanoparticles.

50. The composition of any one of claims 46-49, wherein the superstructure is contained within a suspension.

51. The composition of any one of claims 46-50, wherein each of the noncovalent interactions within the superstructure of nanoparticles is unique.

52. A composition, comprising:
a plurality of superstructures, the superstructures formed from nanoparticles joined in face-to-face contact to form the superstructures, wherein at least 50% of the superstructures comprise at least three nanoparticles and are indistinguishable.

53. The composition of claim 52, wherein at least some of the nanoparticles are nanocubes.

54. The composition of any one of claim 52 or 53, wherein at least some of the superstructures comprise at least ten nanoparticles.

55. The composition of any one of claims 52-54, wherein no more than 50% of the nanoparticles forming the superstructures are identical.

56. The composition of any one of claims 52-55, wherein the superstructures are contained within a suspension.

57. The composition of any one of claims 52-56, wherein the nanoparticles joined in face-to-face contact to form the superstructures are joined by noncovalent interactions.

58. The composition of claim 57, wherein at least some of the noncovalent interactions are nucleic acid interactions.

59. The composition of any one of claim 57 or 58, wherein each of the noncovalent interactions are unique within a superstructure.

60. A composition, comprising:
a suspension comprising a plurality of stable superstructures formed from nanoparticles, wherein at least 30% of the superstructures within the suspension comprise at least three nanoparticles and are indistinguishable.

61. The composition of claim 60, wherein at least 50% of the superstructures within the suspension comprise at least three nanoparticles and are indistinguishable.

62. The composition of any one of claim 60 or 61, wherein at least 90% of the superstructures within the suspension comprise at least three nanoparticles and are indistinguishable.

63. The composition of any one of claims 60-62, wherein at least some of the nanoparticles are nanocubes.

64. The composition of any one of claims 60-63, wherein at least some of the superstructures comprise at least ten nanoparticles.

65. The composition of any one of claims 60-64, wherein no more than 50% of the nanoparticles forming the superstructures are identical.

66. The composition of any one of claims 60-65, wherein the nanoparticles are joined in face-to-face contact to form the superstructures.

67. The composition of claim 66, wherein the nanoparticles are joined by noncovalent interactions.

68. The composition of claim 67, wherein at least some of the noncovalent interactions are nucleic acid interactions.

69. The composition of any one of claim 67 or 68, wherein each of the noncovalent interactions are unique within a superstructure.

70. A composition, comprising:
a first nanoparticle, comprising a first face comprising a first binding partner, a second face comprising a second binding partner, and a third face comprising a third binding partner; and
a second nanoparticle, comprising a first face comprising a binding partner, wherein the binding partner of the second nanoparticle is able to specifically bind to the first binding partner of the first nanoparticle without specifically binding to the second or third binding partners.

71. The composition of claim 70, wherein the first binding partner comprises a first miscibility, the second binding partner comprises a second miscibility incompatible with the first miscibility, and the third binding partner comprises a third miscibility incompatible with the first and second miscibilities.

72. The composition of any one of claim 70 or 71, wherein the first binding partner comprises a first nucleic acid, the second binding partner comprises a second nucleic acid, and the third binding partner comprises a third nucleic acid.

73. The composition of any one of claims 70-72, wherein the nanoparticle is a nanocube.

74. The composition of any one of claims 70-73, wherein the nanoparticle has a largest internal dimension of less than about 1 micrometer.

75. The composition of any one of claims 70-74, wherein the nanoparticles comprise metal.

76. The composition of any one of claims 70-75, wherein the binding partner covers at least 50% of the first face of the nanoparticle.

77. The composition of any one of claims 70-76, wherein the binding partner covers the first face of the nanoparticle.

78. The composition of any one of claims 70-77, wherein the first binding partner comprises a nucleic acid.

79. The composition of any one of claims 70-78, wherein the first binding partner comprises DNA.

80. The composition of any one of claims 70-79, wherein the first binding partner comprises RNA.

81. The composition of any one of claims 70-80, wherein the first binding partner comprises a polymer.

82. The composition of claim 81, wherein the polymer comprises monomers having different hydrophilicities.

83. The composition of any one of claims 70-82, wherein the first binding partner comprises an antibody.

84. The composition of any one of claims 70-83, wherein at least one of the binding partners is able to specifically bind to another binding partner via hydrogen bonds.

85. The composition of any one of claims 70-84, wherein at least one of the binding partners is able to specifically bind to another binding partner via covalent coupling.

86. The composition of any one of claims 70-85, wherein at least one of the binding partners is able to specifically bind to another binding partner via a hydrophobic interaction.

87. A composition, comprising:
a plurality of nanoparticles, comprising at least first and second nanoparticles each comprising faces, the faces of each of the first and second nanoparticles having different arrangements of binding partners, wherein only one face of the first nanoparticle and one face of the second nanoparticle have binding partners that can specifically bind to each other.

88. The composition of claim 87, wherein at least some of the binding partners comprise nucleic acids.

89. The composition of any one of claim 87 or 88, wherein at least some of the nanoparticles are nanocubes.

90. The composition of any one of claims 87-89, wherein the superstructure comprises at least ten nanoparticles.

91. The composition of any one of claims 87-90, wherein the superstructure is contained within a suspension.

92. A device, comprising:
an electronic circuit comprising a conductive pathway defined by a plurality of polyhedral nanoparticles joined in face-to-face contact to form the conductive pathway.

93. An article, comprising:
a superstructure having an interior space, the superstructure formed from a plurality of polyhedral nanoparticles.

94. An article, comprising:
a plurality of nanoparticles positioned to form a superstructure, wherein the superstructure has at least one surface defined by the faces of at least some of the nanoparticles forming the superstructure.

95. An article, comprising:
a sheet formed from a plurality of nanocubes, wherein the sheet has a thickness defined by the thickness of a single nanocube.

96. A method, comprising:
applying a first coating to a first face of a plurality of nanoparticles comprising faces without applying the coating to a second face of the nanoparticles;
applying a second coating to the second face of the nanoparticles without applying the coating to the first face of the nanoparticles; and
enriching the plurality of nanoparticles in nanoparticles having a specific arrangement of the first and second faces.

97. A method of synthesizing a patched nanocube comprising stamping the faces of a nanocube with three or more species of selectively binding patches.

98. A method of synthesizing a superstructure comprising patched nanocubes, comprising combining nanocubes in solution with three or more species of selectively binding chemicals that contain a sequence of regions with different miscible properties.

99. The methods of claim 97 or 98, wherein at least some of the nanocubes comprise metal.

100. The method of claim 99, wherein the metal is selected from the group consisting of Au, Ag, Cu, or Pt.

101. The methods of any one of claims 97-100, wherein at least some of the nanocubes are semiconductors.

102. The method of claim 101, wherein the semiconductors are selected from the group consisting of silicon, copper selenide, copper oxide, or cesium oxide.

103. The methods of any one of claims 97-102, wherein at least some of the nanocubes are magnetic.

104. The method of any one of claims 97-103, wherein at least some of the nanocubes comprise iron oxide.

105. The methods of any one of claims 97-104, wherein at least some of the selectively binding patches comprise DNA and/or RNA.

106. The methods of any one of claims 97-105, wherein at least some of the selectively binding patches comprise antibodies and/or antigens.

107. A method of synthesizing arbitrarily shaped structures comprising combining some combination of nanocubes synthesized using the methods of any one of claims 97-106 in solution and allowing the complimentary selectively binding patches on different nanocubes to bind.

108. The method of claim 107, wherein at least some of the nanocubes bind through hydrogen bonds.

109. The method of any one of claim 107 or 108, wherein at least some of the nanocubes bind through covalent coupling.

110. The method of any one of claims 107-109, wherein at least some of the nanocubes bind through hydrophilic/hydrophobic/fluorinated interactions.

111. The method of any one of claims 107-110, wherein the structure contains an orientation specific patch.

112. The method of any one of claims 107-111, wherein the structure contains a repeating subunit.

113. A method of synthesizing a superstructure, comprising combining nanostructures in solution with three or more species of selectively binding chemicals that contain a sequence of regions with different miscible properties.

114. A method of synthesizing a patched nanostructure comprising stamping the faces of a nanostructure with three or more species of selectively binding patches.
